(19) United States
(12) Patent Application Publication
(10) Pub. No.: US 2025/0027955 A1
(43) Pub. Date: Jan. 23, 2025

(54) ETHYLENE RECEPTORS AND BINDING DOMAINS WITH MODIFIED BINDING KINETICS FOR ETHYLENE

(71) Applicant: Trustees of Dartmouth College, Hanover, NH (US)

(72) Inventors: George Eric Schaller, Lebanon, NH (US); Gevorg Grigoryan, Hanover, NH (US); Beenish Jehan Azhar, Hanover, NH (US)

(73) Assignee: Trustees of Dartmouth College, Hanover, NH (US)

(21) Appl. No.: 18/907,725

(22) Filed: Oct. 7, 2024

Related U.S. Application Data
(63) Continuation of application No. PCT/US2023/017522, filed on Apr. 5, 2023.
(60) Provisional application No. 63/327,469, filed on Apr. 5, 2022.

(51) Int. Cl. G01N 33/68; C07K 14/705; C12N 5/04

(52) U.S. Cl. CPC G01N 33/6872; C07K 14/705; C12N 5/04; C07K 2319/60; C12N 2510/00

(57) ABSTRACT

Embodiments of the present disclosure pertain to an ethylene sensor with an ethylene recognition component and a detecting component. The ethylene recognition component includes at least one ethylene binding domain. The detecting component is operational to generate a detectable signal that correlates to the binding of ethylene to the ethylene binding domain. The ethylene binding domain includes: an Asn residue; at least one variant region that reduces the binding affinity of ethylene to the ethylene receptor when compared to the ethylene binding domain without the variant region; at least one mutation that reduces the binding affinity of ethylene to the ethylene binding domain when compared to the ethylene binding domain without the mutation; or combinations thereof. Additional embodiments pertain to methods of sensing an ethylene from an environment by utilizing the ethylene sensors. Further embodiments pertain to modified cells that include the ethylene binding domains.

### CROSS-REFERENCE TO RELATED APPLICATIONS

The present application is a continuation patent application for International Patent Application No. PCT/US2023/017522, filed on Apr. 5, 2023, which claims the benefit of U.S. Provisional Patent Application No. 63/327,469, filed on Apr. 5, 2022. The entirety of the aforementioned application is incorporated herein by reference.

### STATEMENT REGARDING FEDERALLY SPONSORED RESEARCH

This invention was made with government support under MCB-1517032 and IOS-1856513 awarded by the National Science Foundation. The government has certain rights in the invention.

### STATEMENT UNDER 37 C.F.R. § 1.834(C)(1)

Pursuant to 37 C.F.R. § 1.834, Applicant hereby submits a sequence listing as an XML file (“Sequence Listing”). The name of the file containing the Sequence Listing is “AF60544.P039WO.xml”. The date of the creation of the Sequence Listing is Apr. 5, 2023. The size of the Sequence Listing is 9,000 bytes. Applicant hereby incorporates by reference the material in the Sequence Listing.

### BACKGROUND

A need exists for the development of more effective ethylene sensors that have the capability of continuously monitoring ethylene levels in numerous environments in real-time. Various embodiments of the present disclosure aim to address the aforementioned need.

### SUMMARY

In some embodiments, the present disclosure pertains to an ethylene sensor that includes an ethylene recognition component and a detecting component. In some embodiments, the ethylene recognition component includes at least one ethylene binding domain. In some embodiments, the detecting component is operational to generate a detectable signal that correlates to the binding of ethylene to the ethylene binding domain. In some embodiments, the ethylene binding domain includes: an Asn residue; at least one variant region that reduces the binding affinity of ethylene to the ethylene receptor when compared to the ethylene binding domain without the variant region; at least one mutation that reduces the binding affinity of ethylene to the ethylene binding domain when compared to the ethylene binding domain without the mutation; or combinations thereof.

Additional embodiments of the present disclosure pertain to methods of sensing an ethylene from an environment by (1) exposing the environment to an ethylene sensor of the present disclosure; (2) detecting the presence or absence of a signal from the ethylene sensor; and (3) correlating the absence of the signal to the absence of ethylene in the environment, or correlating the presence of the signal to the presence of ethylene in the environment.

Additional embodiments of the present disclosure pertain to modified cells that include an ethylene binding domain of the present disclosure. In some embodiments, the ethylene binding domain is expressed by at least one of an exogenous gene, a mutated gene, an over-expressed gene, or combinations thereof.

### DESCRIPTION OF THE DRAWINGS

FIG. 1A depicts an ethylene sensor according to an aspect of the present disclosure.

FIG. 1B depicts a method of sensing ethylene according to an aspect of the present disclosure.

FIGS. 2A-2C compare copper and ethylene binding of wild-type and Asp25 mutant versions of ETR1. FIG. 2A shows amino acid conservation of the ethylene-binding domain in ETR1-like proteins. The three conserved transmembrane (TM) helices (H1, HII, and HIII) are indicated. Dark asterisks indicate Cys65 and His69 of ETR1 HII implicated in coordinating the copper cofactor. The light asterisk indicates the highly conserved Asp25 of HI. FIG. 2B shows copper binding of wild-type and Asp25 mutant versions of ETR1 transmembrane domain (ETR1-TMD; n=3). Purified ETR1-TMD was titrated to the BCA₂-Cu(I) complex, and copper binding monitored spectrophotometrically based on the change in absorbance at 562 nm. For comparison, copper binding of a Cys65Ser His69Ala mutation was examined alone and in combination with the Asp25Asn mutation. For site-directed mutations, the single letter abbreviations for the amino acids Asp (D), Ala (A), Asn (N), Cys (C), Glu (E), Gln (Q), His (H), and Ser (S) are used. FIG. 2C shows ethylene binding to yeast transgenically expressing wild-type and Asp25 mutant versions of ETR1. ETR1 protein levels were determined by immunoblot analysis with an anti-ETR1 antibody, and the proteins on the blot staining with Ponceau-S as a loading control. To analyze ethylene binding activity, transgenic yeast samples (n=3; horizontal line=mean) were incubated with 0.21 μL L⁻¹ [¹⁴C]ethylene, in the presence or absence of excess [¹²C]ethylene, the difference between the two values representing the saturable binding; P values for significant saturable binding, as determined by T-test are given for P<0.05.

FIGS. 3A-3D show copper binding by wild-type, Cys65 His69, and Asp25 mutant versions of ETR1. Purified ETR1-TMD was titrated to the BCA₂-Cu(I) complex, and copper binding monitored spectrophotometrically based on the change in absorbance at 562 nm. For comparison, copper binding of a Cys65Ser His69Ala mutation was examined alone and in combination with the Asp25 mutations.

FIGS. 4A-4C show ethylene sensitivity of Arabidopsis seedlings expressing Asp25 mutants of ETR1. Wild-type (wt) and mutant versions of ETR1 were transgenically (tETR1) expressed in the etr1 etr2 ein4 background. FIG. 4A shows ETR1 protein levels as determined by immunoblot analysis with an anti-ETR1 antibody in dark-grown seedlings. BIP serves a loading control. FIG. 4B shows triple-response seedling growth assay to ethylene. Dark-grown seedlings were treated with 0 or 1 μL L⁻¹ ethylene. Images of seedlings are shown along with quantification of the hypocotyl growth response (n≥10; horizontal line=mean), The mutant etr1-1 serves as an ethylene-insensitive control. P values (blue) for significant differences in the ethylene responsiveness of seedlings, as determined by T-test, are given for P<0.05. For data comparison of etr1 etr2 ein4 to wt, tETR1(wt), and tETR1(D25N) lines grown in the absence of ethylene, ANOVA was performed with post-hoc Holm multiple comparison calculation (**P<0.01). FIG. 4C shows ethylene dose response curves of hypocotyl growth in dark grown seedlings for the three lines of ETR1^{st} and ETR1^{D25N} compared to wt and etr1 etr2 ein4. Representative ethylene-insensitive tETR1^{D25A}-#20, tETR1^{D25E}-#19, and tETR1^{D25Q}-#4 lines were also included and examined at 0 and 100 μL L⁻¹ ethylene. The ethylene response is normalized for each line relative to its hypocotyl length at 0 μL L⁻¹ ethylene. (n≥17; SE<6% of hypocotyl length, not shown).

FIGS. 5A-5B show adult plant phenotypes of ETR1^{D25X} mutants. The tETR1^{D25X} plants in the etr1 etr2 ein4 background are compared to the wild type and the etr1 etr2 ein4 mutant. The etr1 etr2 ein4 mutant exhibits reduced growth due to its partial constitutive ethylene-response phenotype, and this can be rescued by transgenic expression of ETR1. The tETR1^{D25X} line numbers are indicated, FIG. 5A shows rosettes of 28-day-old plants. Scale bar=2 cm. FIG. 5B shows adult plants with inflorescences.

FIGS. 6A-6B show short-term ethylene responses of Arabidopsis seedlings expressing Asp25 mutants of ETR1. FIG. 6A shows kinetics of growth response to ethylene of ETR1^{wt} and ETR1^{D25} mutant lines. Ethylene dose response kinetics were analyzed in hypocotyls of 2 days old etiolated seedlings for wild type, the etr1 etr2 ein4 triple mutant, and for the triple mutant complemented with ETR1^{wt} or various ETR1^{D25} mutants as indicated. Measurements were made in air for 1 hr, followed by a 2-hr exposure to 10 μL L⁻¹ ethylene, and then a 5-hr recovery in air. Growth rates for each line are normalized to the growth rate during the first hour in the air. Arrows indicate the time points for the addition and removal of ethylene. Error bars represent SE (n≥7). FIG. 6B shows the effect of ETR1 Asp25 mutants on ethylene-dependent gene expression. Dark-grown seedlings were treated with 0 or 1 μL L⁻¹ ethylene for 2 hr, and gene expression examined by RT-qPCR (n=3). Expression was normalized to a tubulin control and is presented as relative to the untreated wild-type control. Three tETR1-wt and three tETR1^{D25N} lines were examined. The ethylene-insensitive tETR1^{D25A} #20, tETR1^{25E} #19, and tETR1^{D25Q} #4 lines were also included.

FIGS. 7A-7B show kinetics of growth response to ethylene of ETR1^{wt} and ETR1^{D25N} lines. Ethylene dose response kinetics were analyzed in hypocotyls of 2 day old etiolated seedlings for wild type, the etr1 etr2 ein4 triple mutant, and for the triple mutant complemented with ETR1^{wt} (FIG. 7A) or ETR1^{D25N} (FIG. 7B). Measurements were made in air for 1 hr, followed by a 2-hr exposure to 10 μL L⁻¹ ethylene, and then a 5-hr recovery in air. Growth rates for each line are normalized to the growth rate during the first hour in the air. Arrows indicate the time points for the addition and removal of ethylene. Error bars represent SE (n≥9 for all lines, except for ETR1 line 8, n=4).

FIGS. 8A-8C show ethylene binding of Arabidopsis seedlings expressing ETR1^{wt} and ETR1^{D25N}. Two-week-old green seedlings of the ETR1^{wt}-#4 and ETR1^{D25N}-#6 lines expressed in etr1 etr2 ein4 background were examined, as well as the etr1 etr2 ein4 triple mutant itself. FIG. 8A shows representative seedlings (scale bar=1 cm). FIG. 8B shows expression levels of the tETR1 receptor versions as determined by immunoblot, and of ERS1 and ERS2 as determined by qRT-PCR. Significant differences in the saturable binding between lines was determined by ANOVA with post-hoc Holm multiple comparison calculation; different red letters indicate a significant difference at P<0.05. FIG. 8C shows saturable ethylene binding (n=3; error bar=SE) of the seedlings. Seedlings were incubated with 0.31 μL L⁻¹ [¹⁴C]ethylene, in the presence or absence of excess [¹²C]ethylene, the difference between the two values representing the saturable binding. Significant saturable binding as determined by T-test for each line is indicated (blue; *P<0.05, **<0.01, ***<0.001). Significant differences in expression between lines are ANOVA-based analyses (red; P<0.01).

FIGS. 9A-9B show responsiveness of the ETR1^{D25N} mutant to ethylene, silver, and 1-MCP. FIG. 9A shows that the responsiveness of ETR1^{wt} and ETR1^{D25N}, transgenically expressed in the etr1 etr2 ein4 background, is blocked in the presence of silver (Ag). The hypocotyl growth of dark-grown seedlings was analyzed in the absence or presence of 10 μM ethylene (n≥11; error bars=SE). FIG. 9B shows that ETR1^{D25N} responds to 1 μM ethylene in a background lacking all five native ethylene receptors (etr1 etr2 ein4 ers1 ers2), and has this response is blocked by 1 μM 1-MCP (n≥13; error bars=SE). Loss-of-function mutations in the ERS1 and ERS2 genes were generated by a CRISPR-Cas9 approach in the ETR1^{D25N} (etr1 etr2 ein4) line #6. Significant differences in hypocotyl length following treatment were determined for each line by ANOVA with post-hoc Holm multiple comparison calculation; different red letters indicate a significant difference for each line at P<0.05.

FIGS. 10A-10C show the characterization of Lys91 mutants of ETR1. FIG. 10A shows the evolutionary couplings of ETR1 amino acid residues from the ethylene-binding domain, based on the analysis of 1221 ETR1-related sequences using the EV couplings framework. The thickness of the lines represents the strength of individual couplings. The intensity of the blue shading represents the overall strength of couplings for that specific residue. Lys91 and Asp25 are highlighted in red. FIG. 10B shows that ethylene binding to yeast transgenically expressing wild-type and Lys91 mutant versions of ETR1. Immunoblot analysis was used to determine the ETR1 protein levels with an anti-ETR1 antibody: the proteins on the blot were stained with Ponceau-S as a loading control. To determine saturable ethylene binding activity of the ETR1 Lys91 mutants, transgenic yeast samples (n=3) were incubated with 0.21 μL L⁻¹ [¹⁴C]ethylene, in the presence or absence of excess [C]ethylene, the difference between the two values representing the saturable binding; P values for significant saturable binding, as determined by T-test are given for P<0.05, FIG. 10C shows the characterization of Arabidopsis seedlings expressing Lys91 mutants of ETR1. Lys91 mutant versions of ETR1 alone or with Asp25Asn (D25N) were transgenically (tETR1) expressed in the etr1 etr2 ein4 background. Immunoblot analysis was used to determine ETR1 protein levels with an anti-ETR1 antibody in dark-grown seedlings; BIP serves a loading control. For the hypocotyl growth response of dark-grown seedlings to ethylene, seedlings were treated with 0 or 1 μL L⁻¹ ethylene (n≥10; horizontal line=mean). P values (blue) for significant differences in the ethylene responsiveness of seedlings, as determined by T-test, are given for P<0.05.

FIGS. 11A-B show models ETR1 interactions with copper(I) ethylene adducts. FIG. 11A shows Asp-Cys-His (DCII) and Cys-His (CH) models for how Asp25 contributes to copper and ethylene binding and signal transmission via bonding with Lys91. FIG. 11B shows predicted effect of the Asn25 variant on molecular interactions at the ethylene binding site.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “of” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Ethylene detection is important in numerous industries. For instance, ethylene gas detection is important in both the agricultural and petrochemical industries. In particular, the importance of ethylene gas detection to the agricultural and petrochemical industries is due to the role ethylene plays in the ripening and decomposition of fruits and vegetables, as a breakdown product of oil, and the utility of ethylene in the production of such products as polyethylene and polyester.

A variety of electronic sensors have been produced to measure ethylene levels. However, such sensors vary in specificity, sensitivity, the range of ethylene that can be detected, and price.

An interest exists in taking advantage of the natural capability of ethylene receptors to sense ethylene in the design of sensors. For instance, plant ethylene receptors such as ETR1 are extremely sensitive to ethylene and are also able to detect and respond to ethylene across a wide range of concentrations. For these reasons, there is interest in understanding the mechanism by which the receptors bind ethylene and in adapting them for direct use as ethylene sensors.

However, one limitation of utilizing native ethylene receptors is their extremely tight binding of ethylene. For instance, the half-life of many ethylene receptors for ethylene release is approximately 12 hours. Therefore, due to their tight binding, the utilization of many native receptors may not be useful for the continuous and real-time monitoring of ethylene levels.

In sum, a need exists for the development of more effective ethylene sensors that have the capability of continuously monitoring ethylene levels in numerous environments in real-time. Various embodiments of the present disclosure aim to address the aforementioned need.

### Ethylene Sensors

In some embodiments, the present disclosure pertains to ethylene sensors. Ethylene sensors generally include an ethylene recognition component with at least one ethylene binding domain, and a detecting component operational to generate a detectable signal that correlates to the binding of ethylene to the ethylene binding domain.

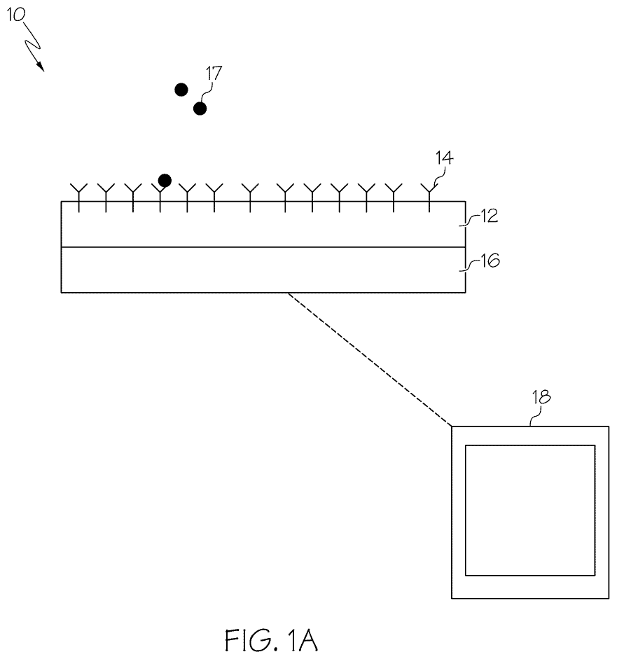

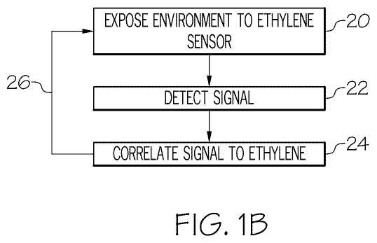

An example of an ethylene sensor is illustrated in FIG. 1A as ethylene sensor 10, which includes an ethylene recognition component 12 with a plurality of ethylene binding domains 14, and a detecting component 16 that is operational to generate a detectable signal that correlates to the binding of ethylene 17 to ethylene binding domains 14. In this Example, ethylene sensor 10 also includes a display 18 for displaying the detectable signal. As set forth in more detail herein, the ethylene sensors of the present disclosure can include numerous embodiments and variations.

### Ethylene Recognition Component

The ethylene sensors of the present disclosure can include numerous types of ethylene recognition components. For instance, in some embodiments, the ethylene recognition component is in the form of a layer. In some embodiments, the ethylene recognition component includes a lipid bilayer. In some embodiments, the ethylene binding domain is embedded in the lipid bilayer. In some embodiments, the ethylene binding domain is present in a whole living cell of prokaryotic or eukaryotic origin.

In some embodiments, the ethylene recognition component includes a cultured cell. In some embodiments, the ethylene binding domain is associated with the cultured cell. For instance, in some embodiments, the ethylene binding domain is embedded with a lipid bilayer of the cultured cell.

In some embodiments, the cultured cell contains or is associated with a detecting component. For instance, in some embodiments, the cultured cell contains at least a portion of a detecting component. In some embodiments, the detecting component includes a tagged gene that is expressed in response to the binding of ethylene to the ethylene binding domain. In some embodiments, the expressed protein of the tagged gene represents a detectable signal that correlates to the binding of ethylene to the ethylene binding domain. In some embodiments, the expressed protein includes a fluorescent protein.

### Ethylene Binding Domains

The ethylene sensors of the present disclosure can also include numerous types of ethylene binding domains. For instance, in some embodiments, the ethylene binding domain represents an isolated fragment of an ethylene receptor. In some embodiments, the isolated fragment is separate and apart from the ethylene receptor. In some embodiments, the isolated fragment is part of a chimeric receptor.

In some embodiments, the ethylene binding domain is part of an ethylene receptor. For instance, in some embodiments, the ethylene binding domain represents an integral part of the ethylene receptor.

In some embodiments, the ethylene binding domain includes: an Asn residue; at least one variant region that reduces the binding affinity of ethylene to the ethylene binding domain when compared to the ethylene binding domain without the variant region; at least one mutation that reduces the binding affinity of ethylene to the ethylene binding domain when compared to the ethylene binding domain without the mutation; or combinations thereof.

In some embodiments, the ethylene binding domain includes an Asn residue. In some embodiments, the Asn residue represents a variant of the ethylene binding domain. In some embodiments, the Asn residue represents a mutation of the ethylene binding domain. In some embodiments, the mutation represents a mutation from Asp to Asn.

In some embodiments, the ethylene binding domain includes at least one variant region that reduces the binding affinity of ethylene to the ethylene binding domain when compared to the ethylene binding domain without the variant region. In some embodiments, the variant region includes an Asn residue.

In some embodiments, the ethylene binding domain includes at least one mutation that reduces the binding affinity of ethylene to the ethylene binding domain when compared to the ethylene binding domain without the mutation. In some embodiments, the mutation includes an Asn residue. In some embodiments, the mutation represents a mutation from Asp to Asn.

In some embodiments, the mutation includes a mutation on a copper binding domain of the ethylene recognition component. In some embodiments, the mutation on the copper binding domain of the ethylene recognition component reduces the binding affinity of copper to the copper binding domain when compared to the binding affinity of copper to the copper binding domain without the mutation.

Ethylene binding domains may be part of, or derived from, various ethylene receptors. For instance, in some embodiments, the ethylene receptor includes, without limitation, ETR1, ETR2, EIN4, ERS1, ERS2 (e.g., as described for the plant Arabidopsis thaliana), ERS1, ERS2, ETR2, ETR3, ETR4 (e.g., as described for the plant Oryza sativa), SynSLR1212 (SynETR1) of the cyanobacterium Synechocystis sp. PCC6803, analogs thereof, homologs thereof, or combinations thereof (e.g., as identified in prokaryotic and/or eukaryotic species). In some embodiments, the ethylene receptor includes ETR1.

In some embodiments, the ethylene receptor originates from at least one of Arabidopsis thaliana, Oryza sativa, cyanobacteria (e.g., Synechocystis sp. PCC6803, Stenomitos frigidus, Gloeocapsa sp. PCC 73106, and/or Stanieria sp. NIES-3757), or combinations thereof. In some embodiments, the ethylene receptor originates from Arabidopsis thaliana.

In some embodiments, the ethylene receptor includes an analog of an ethylene receptor (e.g., any of the aforementioned ethylene receptors). In some embodiments, the analog is at least 40% identical to the ethylene receptor. In some embodiments, the analog is at least 50% identical to the ethylene receptor. In some embodiments, the analog is at least 60% identical to the ethylene receptor. In some embodiments, the analog is at least 70% identical to the ethylene receptor. In some embodiments, the analog is at least 75% identical to the ethylene receptor. In some embodiments, the analog is at least 80% identical to the ethylene receptor. In some embodiments, the analog is at least 85% identical to the ethylene receptor. In some embodiments, the analog is at least 90% identical to the ethylene receptor. In some embodiments, the analog is at least 95% identical to the ethylene receptor. In some embodiments, the analog is at least 99% identical to the ethylene receptor.

In some embodiments, the ethylene receptor includes a homolog of an ethylene receptor (e.g., any of the aforementioned ethylene receptors). In some embodiments, the homolog is at least 40% identical to the ethylene receptor. In some embodiments, the homolog is at least 50% identical to the ethylene receptor. In some embodiments, the homolog is at least 60% identical to the ethylene receptor. In some embodiments, the homolog is at least 70% identical to the ethylene receptor. In some embodiments, the homolog is at least 75% identical to the ethylene receptor. In some embodiments, the homolog is at least 80% identical to the ethylene receptor. In some embodiments, the homolog is at least 85% identical to the ethylene receptor. In some embodiments, the homolog is at least 90% identical to the ethylene receptor. In some embodiments, the homolog is at least 95% identical to the ethylene receptor. In some embodiments, the homolog is at least 99% identical to the ethylene receptor.

In some embodiments, the ethylene receptor includes SEQ ID NO: 1. In some embodiments, the ethylene receptor shares at least 40% sequence identity to SEQ ID NO: 1. In some embodiments, the ethylene receptor shares at least 50% sequence identity to SEQ ID NO: 1. In some embodiments, the ethylene receptor shares at least 60% sequence identity to SEQ ID NO: 1. In some embodiments, the ethylene receptor shares at least 70% sequence identity to SEQ ID NO: 1. In some embodiments, the ethylene receptor shares at least 75% sequence identity to SEQ ID NO: 1. In some embodiments, the ethylene receptor shares at least 80% sequence identity to SEQ ID NO: 1. In some embodiments, the ethylene receptor shares at least 85% sequence identity to SEQ ID NO: 1. In some embodiments, the ethylene receptor shares at least 90% sequence identity to SEQ ID NO: 1. In some embodiments, the ethylene receptor shares at least 95% sequence identity to SEQ ID NO: 1. In some embodiments, the ethylene receptor shares at least 99% sequence identity to SEQ ID NO: 1.

In some embodiments, the ethylene binding domain includes SEQ ID NO: 2. In some embodiments, the ethylene binding domain shares at least 40% sequence identity to SEQ ID NO: 2. In some embodiments, the ethylene binding domain shares at least 50% sequence identity to SEQ ID NO: 2. In some embodiments, the ethylene binding domain shares at least 60% sequence identity to SEQ ID NO: 2. In some embodiments, the ethylene binding domain shares at least 70% sequence identity to SEQ ID NO: 2. In some embodiments, the ethylene binding domain shares at least 75% sequence identity to SEQ ID NO: 2. In some embodiments, the ethylene binding domain shares at least 80% sequence identity to SEQ ID NO: 2. In some embodiments, the ethylene binding domain shares at least 85% sequence identity to SEQ ID NO: 2. In some embodiments, the ethylene binding domain shares at least 90% sequence identity to SEQ ID NO: 2. In some embodiments, the ethylene binding domain shares at least 95% sequence identity to SEQ ID NO: 2. In some embodiments, the ethylene binding domain shares at least 99% sequence identity to SEQ ID NO: 2.

In some embodiments, the ethylene binding domain includes SEQ ID NO: 3. In some embodiments, the ethylene binding domain shares at least 40% sequence identity to SEQ ID NO: 3. In some embodiments, the ethylene binding domain shares at least 50% sequence identity to SEQ ID NO: 3. In some embodiments, the ethylene binding domain shares at least 60% sequence identity to SEQ ID NO: 3. In some embodiments, the ethylene binding domain shares at least 70% sequence identity to SEQ ID NO: 3. In some embodiments, the ethylene binding domain shares at least 75% sequence identity to SEQ ID NO: 3. In some embodiments, the ethylene binding domain shares at least 80% sequence identity to SEQ ID NO: 3. In some embodiments, the ethylene binding domain shares at least 85% sequence identity to SEQ ID NO: 3. In some embodiments, the ethylene binding domain shares at least 90% sequence identity to SEQ ID NO: 3. In some embodiments, the ethylene binding domain shares at least 95% sequence identity to SEQ ID NO: 3. In some embodiments, the ethylene binding domain shares at least 99% sequence identity to SEQ ID NO: 3.

In some embodiments, the ethylene binding domain includes SEQ ID NO: 4. In some embodiments, the ethylene binding domain shares at least 40% sequence identity to SEQ ID NO: 4. In some embodiments, the ethylene binding domain shares at least 50% sequence identity to SEQ ID NO: 4. In some embodiments, the ethylene binding domain shares at least 60% sequence identity to SEQ ID NO: 4. In some embodiments, the ethylene binding domain shares at least 70% sequence identity to SEQ ID NO: 4. In some embodiments, the ethylene binding domain shares at least 75% sequence identity to SEQ ID NO: 4. In some embodiments, the ethylene binding domain shares at least 80% sequence identity to SEQ ID NO: 4. In some embodiments, the ethylene binding domain shares at least 85% sequence identity to SEQ ID NO: 4. In some embodiments, the ethylene binding domain shares at least 90% sequence identity to SEQ ID NO: 4. In some embodiments, the ethylene binding domain shares at least 95% sequence identity to SEQ ID NO: 4. In some embodiments, the ethylene binding domain shares at least 99% sequence identity to SEQ ID NO: 4.

In some embodiments, the ethylene binding domain includes SEQ ID NO: 5. In some embodiments, the ethylene binding domain shares at least 40% sequence identity to SEQ ID NO: 5. In some embodiments, the ethylene binding domain shares at least 50% sequence identity to SEQ ID NO: 5. In some embodiments, the ethylene binding domain shares at least 60% sequence identity to SEQ ID NO: 5. In some embodiments, the ethylene binding domain shares at least 70% sequence identity to SEQ ID NO: 5. In some embodiments, the ethylene binding domain shares at least 75% sequence identity to SEQ ID NO: 5. In some embodiments, the ethylene binding domain shares at least 80% sequence identity to SEQ ID NO: 5. In some embodiments, the ethylene binding domain shares at least 85% sequence identity to SEQ ID NO: 5. In some embodiments, the ethylene binding domain shares at least 90% sequence identity to SEQ ID NO: 5. In some embodiments, the ethylene binding domain shares at least 95% sequence identity to SEQ ID NO: 5. In some embodiments, the ethylene binding domain shares at least 99% sequence identity to SEQ ID NO: 5.

The ethylene binding domains of the present disclosure can have various modified binding affinities for ethylene. For instance, in some embodiments, the ethylene binding domain has a half-life for ethylene release of less than 12 hours. In some embodiments, the ethylene binding domain has a half-life for ethylene release of less than 10 hours. In some embodiments, the ethylene binding domain has a half-life for ethylene release of less than 6 hours. In some embodiments, the ethylene binding domain has a half-life for ethylene release of less than 2 hours. In some embodiments, the ethylene binding domain has a half-life for ethylene release of less than an hour. In some embodiments, the ethylene binding domain has a half-life for ethylene release of less than 30 minutes. In some embodiments, the ethylene binding domain has a half-life for ethylene release of less than 15 minutes. In some embodiments, the ethylene binding domain has a half-life for ethylene release of less than 10 minutes. In some embodiments, the ethylene binding domain has a half-life for ethylene release of less than 5 minutes.

### Detecting Component

Detecting components generally refer to ethylene sensor components that are operational to generate a detectable signal that correlates to the binding of ethylene to ethylene binding domains. The ethylene sensors of the present disclosure can also include numerous types of detecting components.

For instance, in some embodiments, the detecting component is in the form of a layer. In some embodiments, the detecting component is in the form of an electrode.

In some embodiments, the detecting component includes a physicochemical transducer. In some embodiments, the physicochemical transducer is operational for converting at least one physicochemical change produced from the binding of ethylene to the ethylene binding domain to a detectable signal. In some embodiments, the physicochemical transducer includes, without limitation, an amperometric transducer, a potentiometric transducer, an impedance-based transducer, a conductometric-based transducer, an optical biotransducer, a field-effect transistor, a gravimetric transducer, a pyroelectric transducer, or combinations thereof.

In some embodiments, the detecting components of the present disclosure include a component of a cultured cell. For instance, in some embodiments, the detecting component includes a tagged gene that is expressed in response to the binding of ethylene to the ethylene binding domain. In some embodiments, the expressed protein of the tagged gene represents a detectable signal that correlates to the binding of ethylene to the ethylene binding domain. In some embodiments, the expressed protein includes a fluorescent protein.

The detecting components of the present disclosure may be operational to generate various types of detectable signals. For instance, in some embodiments, the detectable signal includes, without limitation, an optical signal, an electrical signal, a fluorescent signal, or combinations thereof. In some embodiments, the detectable signal is proportionate to the concentration of the ethylene. In some embodiments, the detectable signal is detectable in real-time.

### Structures and Additional Components

The ethylene sensors of the present disclosure can include numerous structures and additional components. For instance, in some embodiments, the detecting component is coupled to the ethylene recognition component. In some embodiments, the detecting component is in electrical communication with the ethylene recognition component.

In some embodiments, the ethylene sensors of the present disclosure also include a display (e.g., display 18 shown in FIG. 1A). In some embodiments, the display is operational for displaying the detectable signal. In some embodiments, the display includes a digital screen, such as a computer screen. In some embodiments, the display is in electronic communication with the detecting component.

### Attributes

The ethylene sensors of the present disclosure may have various advantageous attributes. For instance, in some embodiments, the ethylene sensors of the present disclosure are operational to continuously sense ethylene in real-time.

The ethylene sensors of the present disclosure may also be operational to detect various types of ethylene. For instance, in some embodiments, the detectable ethylene is in the form of a gas.

### Methods of Sensing Ethylene

Additional embodiments of the present disclosure pertain to methods of sensing ethylene from an environment. In some embodiments illustrated in FIG. 1B, the methods of the present disclosure include: exposing the environment to a sensor of the present disclosure (step 20); detecting the presence or absence of a signal from the sensor (step 22); and correlating the absence of the signal to the absence of ethylene in the environment, or correlating the presence of the signal to the presence of ethylene in the environment (step 24). In some embodiments, the ethylene sensing methods of the present disclosure may be repeated in order to continuously sense ethylene in the environment (step 26).

The methods of the present disclosure may utilize various sensors. Suitable sensors include the sensors of the present disclosure, as described supra.

The methods of the present disclosure may be utilized to sense ethylene from various environments. For instance, in some embodiments, the environment includes an agricultural environment. In some embodiments, the environment includes a petrochemical environment. In some embodiments, the environment includes a research laboratory environment.

Additionally, the methods of the present disclosure may be utilized to sense various types of ethylene from an environment. For instance, in some embodiments, the ethylene is in the form of a gas.

The methods of the present disclosure may sense ethylene in various manners. For instance, in some embodiments, the sensing occurs continuously. In some embodiments, the sensing occurs in real-time. In some embodiments, the sensing occurs continuously and in real-time.

The methods of the present disclosure can have various advantageous applications. For instance, in some embodiments, the methods of the present disclosure may be utilized to monitor the ripening of fruits and/or vegetables. In some embodiments, the methods of the present disclosure may be utilized to avoid the decomposition of the fruits and/or vegetables. In some embodiments, the methods of the present disclosure may be utilized to monitor the release of ethylene from a petrochemical plant.

### Modified Cells Expressing Ethylene Binding Domains

Additional embodiments of the present disclosure pertain to modified cells that include at least one ethylene binding domain of the present disclosure. In some embodiments, the ethylene binding domain is expressed by at least one of an exogenous gene, a mutated gene, an over-expressed gene, or combinations thereof.

The modified cells of the present disclosure can include various ethylene binding domains. Suitable binding domains were described supra. For instance, in some embodiments, the ethylene binding domain of the modified cells of the present disclosure include: an Asn residue; at least one variant region that reduces the binding affinity of ethylene to the ethylene binding domain when compared to the ethylene binding domain without the variant region; at least one mutation that reduces the binding affinity of ethylene to the ethylene binding domain when compared to the ethylene binding domain without the mutation; or combinations thereof.

In some embodiments, the ethylene binding domain includes an Asn residue. In some embodiments, the Asn residue represents a variant of the ethylene binding domain. In some embodiments, the Asn residue represents a mutation of the ethylene binding domain. In some embodiments, the mutation represents a mutation from Asp to Asn.

In some embodiments, the ethylene binding domain includes at least one variant region that reduces the binding affinity of ethylene to the ethylene binding domain when compared to the ethylene binding domain without the variant region. In some embodiments, the variant region includes an Asn residue.

In some embodiments, the ethylene binding domain includes at least one mutation that reduces the binding affinity of ethylene to the ethylene binding domain when compared to the ethylene binding domain without the mutation. In some embodiments, the mutation includes an Asn residue. In some embodiments, the mutation represents a mutation from Asp to Asn. In some embodiments, the mutation includes a mutation on a copper binding domain of the ethylene recognition component, In some embodiments, the mutation reduces the binding affinity of copper to the copper binding domain when compared to the binding affinity of copper to the copper binding domain without the mutation.

In some embodiments, the ethylene binding domain of the modified cells of the present disclosure is expressed as a component of an ethylene receptor. In some embodiments, the ethylene binding domain is expressed as a fragment that is separate and apart from an ethylene receptor. In some embodiments, the ethylene binding domain is expressed as part of a chimeric receptor.

In some embodiments, the ethylene binding domain includes SEQ ID NO: 2 or a sequence with at least 40% sequence identity, 50% sequence identity, 60% sequence identity, 70% sequence identity, 75% sequence identity, 80% sequence identity, 85% sequence identity, 90% sequence identity, 95% sequence identity or 99% sequence identity to SEQ ID NO: 2. In some embodiments, the ethylene binding domain includes SEQ ID NO: 2.

In some embodiments, the ethylene binding domain includes SEQ ID NO: 3 or a sequence with at least 40% sequence identity, 50% sequence identity, 60% sequence identity, 70% sequence identity, 75% sequence identity, 80% sequence identity, 85% sequence identity, 90% sequence identity, 95% sequence identity or 99% sequence identity to SEQ ID NO: 3. In some embodiments, the ethylene binding domain includes SEQ ID NO: 3.

In some embodiments, the ethylene binding domain includes SEQ ID NO: 4 or a sequence with at least 40% sequence identity, 50% sequence identity, 60% sequence identity, 70% sequence identity, 75% sequence identity, 80% sequence identity, 85% sequence identity, 90% sequence identity, 95% sequence identity or 99% sequence identity to SEQ ID NO: 4. In some embodiments, the ethylene binding domain includes SEQ ID NO: 4.

In some embodiments, the ethylene binding domain includes SEQ ID NO: 5 or a sequence with at least 40% sequence identity, 50% sequence identity, 60% sequence identity, 70% sequence identity, 75% sequence identity, 80% sequence identity, 85% sequence identity, 90% sequence identity, 95% sequence identity or 99% sequence identity to SEQ ID NO: 5. In some embodiments, the ethylene binding domain includes SEQ ID NO: 5.

In some embodiments, the ethylene receptor includes, without limitation, ETR1, ETR2, EIN4, ERS1, ERS2, ERS1, ERS2, ETR2, ETR3, ETR4, SynSLR1212 (SynETR1) of the cyanobacterium Synechocystis sp. PCC6803, analogs thereof, homologs thereof, or combinations thereof. In some embodiments, the ethylene receptor originates from at least one of Arabidopsis thaliana, Oryza sativa, cyanobacteria, or combinations thereof. In some embodiments, the ethylene receptor includes ETR1.

In some embodiments, the ethylene receptor includes SEQ ID NO: 1 or a sequence with at least 40% sequence identity, 50% sequence identity, 60% sequence identity, 70% sequence identity, 75% sequence identity, 80% sequence identity, 85% sequence identity, 90% sequence identity, 95% sequence identity or 99% sequence identity to SEQ ID NO: 1. In some embodiments, the ethylene receptor includes SEQ ID NO: 1.

In some embodiments, the modified cell includes a bacterial cell. In some embodiments, the modified cell includes a plant cell. In some embodiments, the modified cell includes a seed cell.

### Modified Plants or Seeds Expressing Ethylene Binding Domains

Further embodiments of the present disclosure pertain to modified plants or seeds that include the modified cells of the present disclosure. For instance, in some embodiments, the modified plants or seeds include an Arabidopsis thaliana plant that includes an ethylene binding domain of the present disclosure.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. An Aspartate Residue of the Ethylene Receptor ETR1 Mediates High-Affinity Ethylene Binding

The gaseous hormone ethylene regulates multiple aspects of plant growth and development, ripening being the best known of these, as well as responses to biotic and abiotic factors. Ethylene is perceived in plants by membrane-bound receptors, the first identified and best studied of these being ETR1 from Arabidopsis. Most plants contain families of ethylene receptors, the five-member ethylene-receptor family of Arabidopsis consisting of ETR1, ETR2, EIN4, ERS1, and ERS2. The plant ethylene receptors have similar overall structures with transmembrane (TM) domains near their N-termini and signaling motifs in their C-terminal regions. The N-terminal TM domains contain the ethylene-binding site, and also serve in membrane localization of the receptor, the majority of the receptors being found associated with the endoplasmic reticulum.

Following the TM domain is a GAF domain implicated in receptor interactions. The C-terminal portions of each receptor contain domains with similarity to histidine kinases and in some cases the receiver domains of response regulators. Histidine kinases and receiver domains are signaling elements originally identified as components in bacterial phosphorelays and are now known to be present in plants, fungi, and slime molds. The plant ethylene receptors are negative regulators of ethylene signal transduction, such that the receptors are ‘on’ in the absence of ethylene and actively repress the ethylene response, and ‘off’ when bound to ethylene, allowing for de-repression of the ethylene response. Since the initial identification of ETR1 in plants, similar proteins with the conserved features of the ethylene-binding domain (EBD) have also been identified in prokaryotes, notably in cyanobacteria.

Due in part to the difficulty in obtaining high-resolution structural information from TM domains, much of what is known about the requirements for ethylene binding by the receptors comes from a coupling of biochemical and genetic analyses. Through these analyses, the receptors have been determined to function as homodimers, with ethylene binding mediated through an associated Cu(I) co-factor. A set of highly conserved Cys and His residues in the TM domain is implicated in chelating the copper cofactor.

Initial analysis indicated the existence of one copper cofactor per receptor dimer, suggesting a model in which the copper is chelated by two Cys and two His residues, thereby resulting in a single ethylene binding site per receptor dimer. However, recent analysis is consistent with the existence of one copper cofactor per receptor monomer, which supports a model with two copper cofactors and therefore two ethylene binding sites per receptor dimer. The well-characterized missense mutation etr1-1 arises due to a mutation in the liganding Cys residue (Cys65Tyr), resulting in a receptor that no longer binds the copper cofactor and as a result also no longer binds ethylene. The etr1-1 mutation confers dominant ethylene insensitivity on plants because of this inability to perceive the ethylene signal. Additional missense mutations in the receptor have further refined an understanding of ethylene binding and signal transduction, as has computational modeling and tryptophan scanning mutagenesis.

A major and still unresolved question is how the ethylene receptors bind ethylene with such high affinity. Ethylene binds to ETR1 with a calculated dissociation constant (K_{d}) of 2.4×10⁻⁹ M, and with a half-life for dissociation of over 12 hours, consistent with plants responding to ethylene concentrations as low as 0.2 nL L⁻¹.

In this Example, Applicant describes the identification of a highly conserved aspartate within the ETR1 TM domain (Asp25) as playing a critical role in copper and ethylene binding. Applicant determined that a natural variant of Asp25 (Asp25Asn) is still functional but has a reduced affinity for ethylene, pointing to the key role Asp25 plays in modulating high-affinity ethylene binding by the receptors. Additionally, Applicant has identified a highly conserved lysine residue (Lys91) that may form a polar bridge to Asp25 to internally transduce the ethylene signal within the receptor to mediate changes in signaling output.

### Example 1.1. A Highly Conserved Asp Residue in the EBD Modulates Copper and Ethylene Binding by ETR1

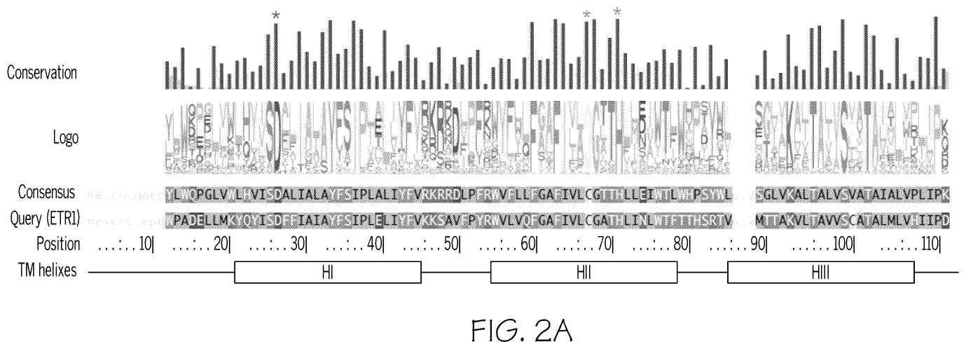

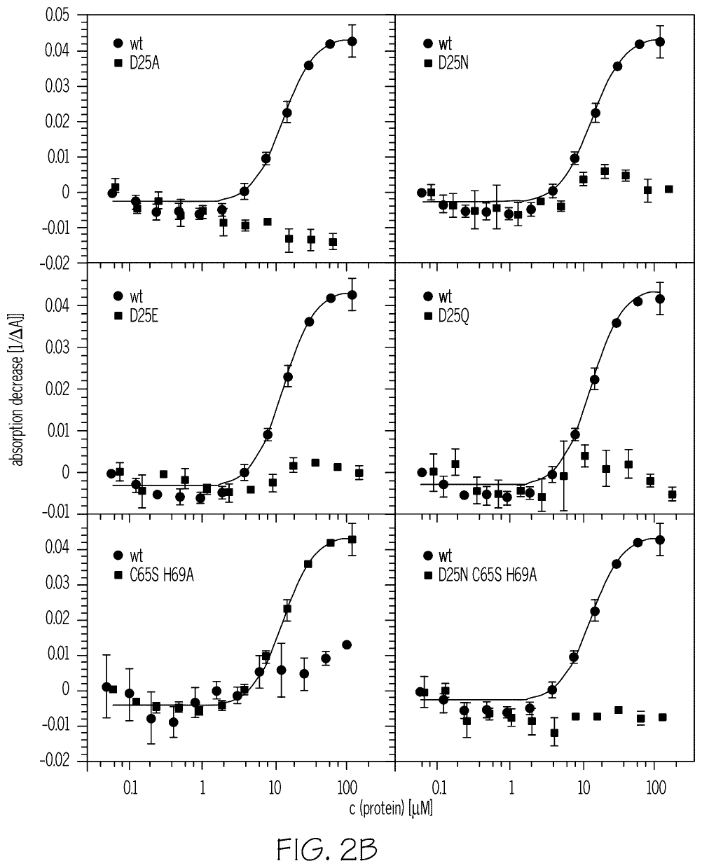

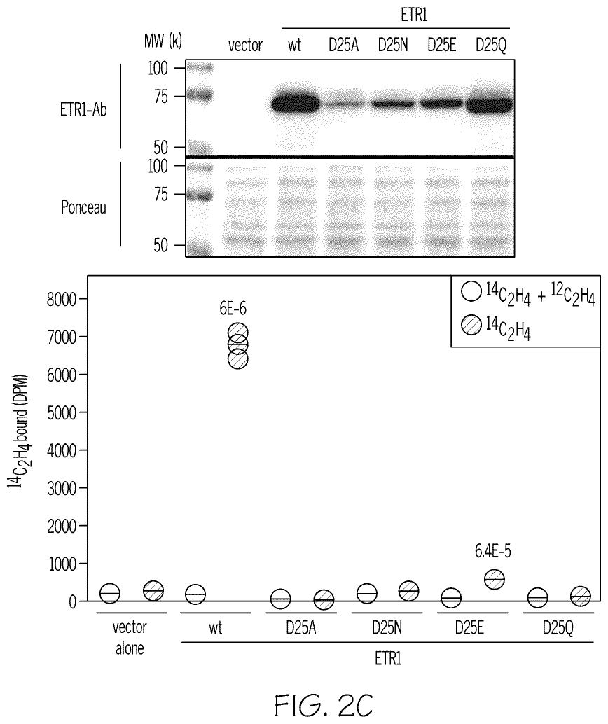

The EBD of the receptor ETR1 of Arabidopsis is contained within the N-terminal transmembrane domain of the protein. This transmembrane domain contains three predicted transmembrane helixes, with the Cys65 and a His69 residues of transmembrane helix II directly implicated in coordinating the copper cofactor required for ethylene binding. Similar EBDs have been identified in a wide variety of organisms, including prokaryotes, the ethylene-binding capability of receptors from Arabidopsis, tomato, and the cyanobacterium Synechocystis sp PCC 6803 having all been confirmed. FIG. 2A indicates the degree of amino-acid conservation found in a comparison of EBDs from 1221 eukaryotic and prokaryotic sequences related to ETR1. As predicted, the Cys and His residues of TMII implicated in coordinating the copper cofactor of ETR1 are highly conserved in EBDs.

Of particular interest are the additional conserved polar and charged residues found in the TM helixes, such as Asp25 (D25) of helix I in ETR1 (FIG. 2A), because such residues are likely to play significant roles in ethylene binding and/or signal transduction. Mutation of Asp25 to Ala (D25A) abolishes ethylene binding by the receptor when analyzed in a heterologous yeast expression system and also confers dominant ethylene-insensitivity when expressed in Arabidopsis. Computational modeling places Asp25 of helix I in proximity to Cys65 and His69 of helix II, suggesting that it could play a role in coordinating the copper cofactor. Interestingly, although Asp is found in 93.55% of the sequences examined, in some cases (3.67%) it is substituted by an Asn residue, most commonly in cyanobacteria but also in several plants, Pyrus communis (Pear) and Cajanus cajan (Pigeon pea).

To characterize the role of Asp25 in copper and ethylene binding, Applicant generated four site-directed mutant versions of ETR1. ETR1^{D25A} was previously found to eliminate ethylene binding. ETR1^{D25N} represents a relatively conserved change of the Asp R-group from a carboxylic acid to a carboxamide, one that will preserve the general size of the side group but which eliminates the negative charge. As noted above, although Asp25 is highly conserved in EBDs, an Asn residue is found at that position in a few EBDs (FIG. 2A). ETR1^{D25E} preserves the carboxylic acid and its negative charge found in the Asp R-group, but Glu has a longer sidechain than does Asp. The ETR1^{D25Q} mutation is analogous to that for ETR1^{D25N}, exchanging a carboxyamide for a carboxylic acid on the R-group of Glu, and so eliminating the negative charge of the Glu.

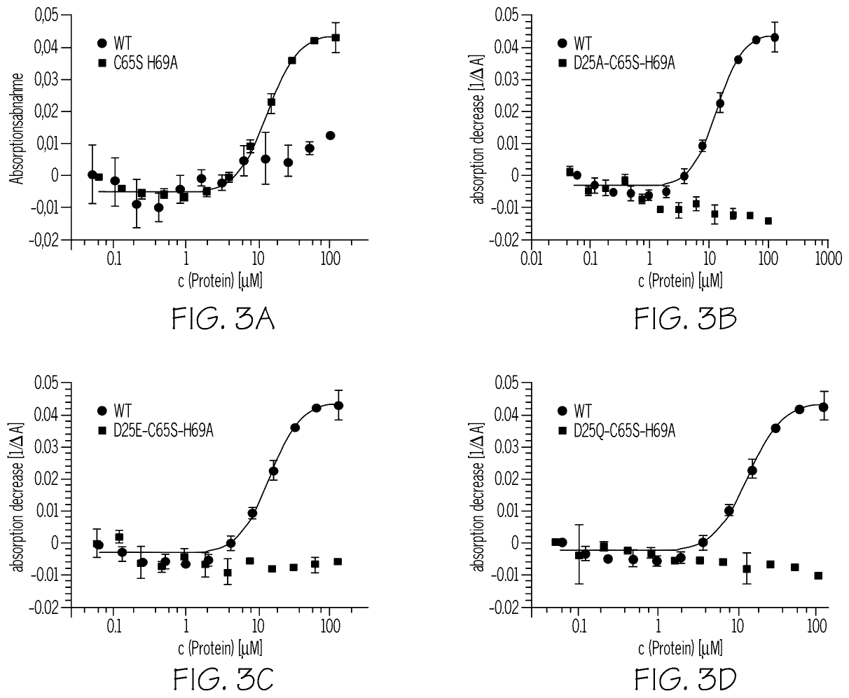

Applicant tested the effects of Asp25 mutants on copper binding to the ETR1 transmembrane domain following expression and purification from E. coli. Cu(I) was stabilized by the copper chelator BCA, and then titrated with increasing ETR1 protein concentration. As shown in FIG. 2B, titration with ETR1^{wt} results in copper binding and a concomitant decrease in absorbance at 562 nm of the purple BCA₂-Cu(I) complex. In contrast, no copper binding was observed for ETR1^{D25A} and only minimal residual binding observed for ETR1^{D25N}, ETR1^{D25E} and ETR1^{D25Q} (FIG. 2B). These data thus support a model in which Asp25 contributes to copper binding. Furthermore, Applicant found that the Asp25 mutants had an additive effect on copper binding when combined with an ETR1^{C65S;H69A} mutant, because the ETR1^{C65S;H69A} mutant exhibited residual binding that was eliminated when combined with the Asp25 mutations (ETR1^{D25X;C65S;H69A}) (FIG. 2B and FIGS. 3A-D).

Saturable ethylene binding of the ETR1 Asp25 mutants was examined by heterologous expression in yeast, with binding to [¹⁴C]ethylene determined in the presence or absence of excess [¹²C]ethylene (FIG. 2C). Saturable binding of [¹⁴C]ethylene was observed for ETR1^{wt}, as the positive control, and not with the pYCDE2 vector negative control (FIG. 2C). Binding of ethylene by ETR1^{D25E} was still observed but was substantially reduced compared to that observed with ETR1^{wt}, likely arising due to steric problems from the larger size of the Glu R-group as well as differences in protein expression levels (FIG. 2C). No saturable [¹⁴C]ethylene binding was detected for the other D25 mutants. This included the ETR1^{D25A} mutant, consistent with previous observations, as well as for the ETR1^{D25N} and ETR1^{D25Q} mutants (FIG. 2C). Results from the ethylene binding assay are therefore consistent with the negative charge at D25 playing a significant role in mediating high-affinity ethylene binding.

### Example 1.2. Functional Analysis of Asp25 Mutations on the ETR1 Responses in Planta

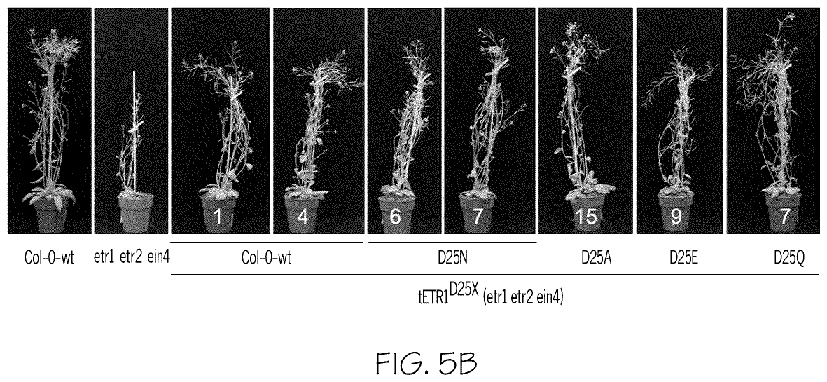

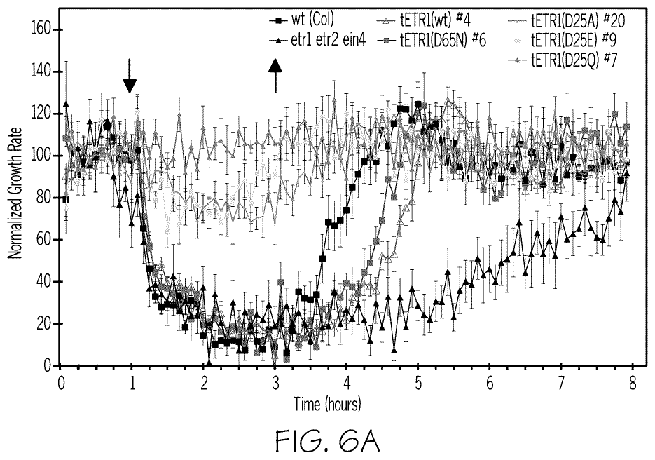

Functionality of the ETR1 Asp25 mutants was tested by transgenic expression in the etr1 etr2 ein4 Arabidopsis background. The rationale for this approach is that the etr1 etr2 ein4 triple mutant exhibits a partial constitutive ethylene-response phenotype, resulting in reduced shoot growth as well as a shorter hypocotyl in the air than is found in the wild type (FIGS. 3A-3B and FIGS. 5A-5B).

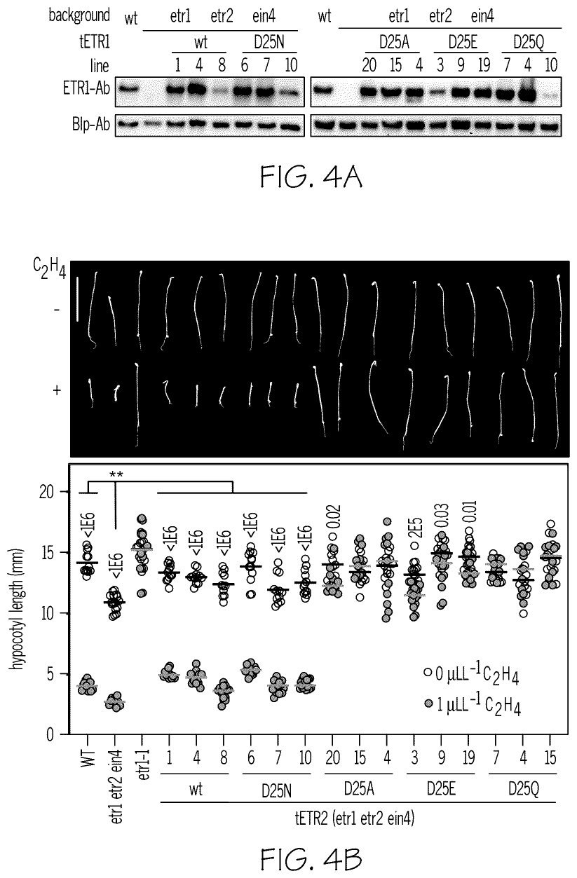

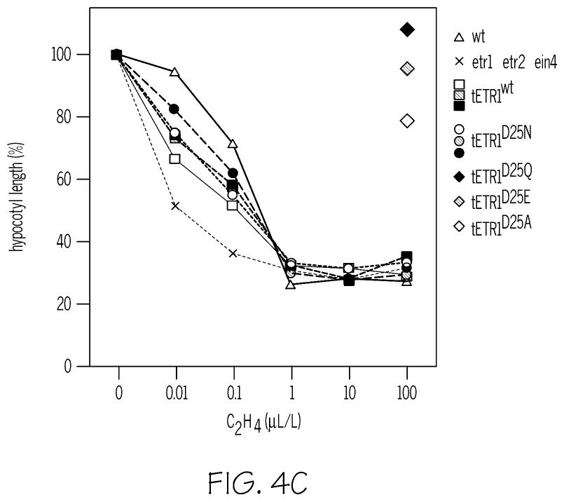

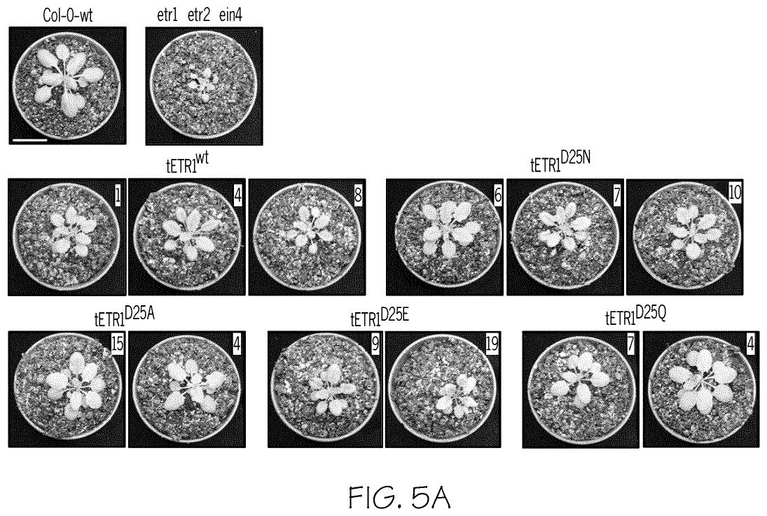

Applicant can thus exploit the triple mutant to assess the ability of the ETR1 transgenes to rescue growth in the absence of ethylene as well as their ability to mediate a response to ethylene. Such an analysis indicates whether the encoded receptors are able to assume the ‘on’ conformation that represses the ethylene response in air, as well as the ‘off’ conformation that occurs in response to ethylene binding. All transgenes were expressed based on immunological detection of the tETR1 protein, and all rescued growth of the etr1 etr2 ein4 triple mutant in air based on hypocotyl and adult shoot growth analysis, indicating that all the tETR1 proteins (tETR1^{wt}, tETR1^{D25N}, tETR1^{D25Q}, tETR1^{D25E}, tETR1^{D25A}) are capable of assuming the ‘on’ conformation (FIGS. 4A-4B, and FIGS. 5A-5B).

Previous studies have found that the site-directed mutation of residues that result in a loss of ethylene-binding activity typically confer dominant ethylene insensitivity on the seedlings, due to an inability of the etr1 mutant protein to switch from its ‘on’ to its ‘off’ conformation. Based on this, Applicant anticipated that those Asp25 mutants that resulted in a loss of high-affinity ethylene binding (tETR1^{D25N}, tETR1^{D25Q}, and tETR1^{D25A}) would confer dominant ethylene insensitivity. Since the tETR1^{D25E} protein still exhibited reduced ethylene binding, it was possible that it would respond similarly to wild type, but it was also possible that tETR1^{D25E} would confer dominant insensitivity due to the reduced ability to bind and/or the perturbation of the ethylene binding site.

As shown in FIGS. 4A-4B, the ETR1^{D25A}, ETR1^{D25E}, and ETR1^{D25Q} transgenes all conferred ethylene insensitivity based on a hypocotyl growth response analysis, consistent with predictions. Surprisingly, the ETR1^{D25N} transgene rescued the hypocotyl growth response to ethylene to the same degree as ETR1^{wt} even though, based on Applicant's previous analyses, ETR1^{D25N} was compromised in its copper and ethylene binding ability. A hypocotyl dose response analysis, spanning 0 to 100 μL/L ethylene, confirmed a similar ethylene responsiveness for both the ETR1^{wt} and ETR1^{D25N} lines (FIG. 4C).

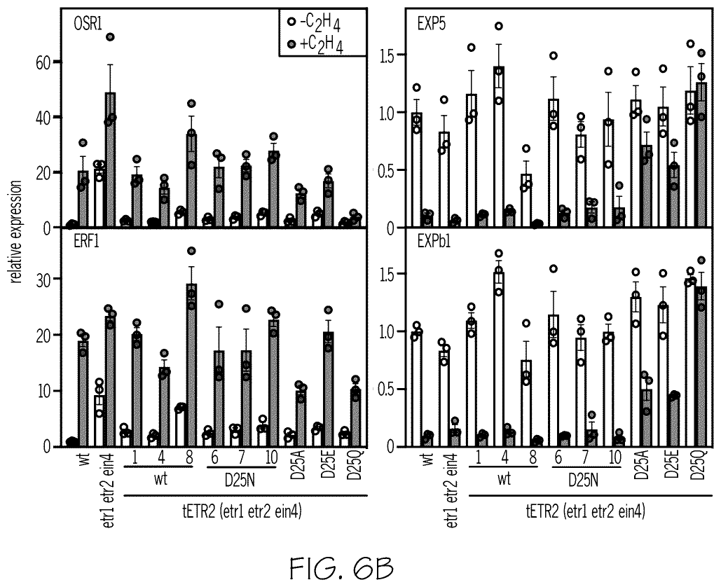

Based on the differences Applicant uncovered for the long-term ethylene growth responses for the Asp25 mutants, Applicant also analyzed their short-term hypocotyl growth and molecular responses to ethylene. To this end, Applicant performed a short-term kinetic analysis, analyzing the initial hypocotyl growth inhibition in response to 10 μL/L ethylene and the growth recovery following the removal of ethylene after two hours of treatment (FIG. 6A). As shown in FIG. 6A, both wild type and the triple mutant etr1 etr2 ein4 exhibit a rapid inhibition of hypocotyl growth in response to ethylene, but the growth recovery for the etr1 etr2 ein4 mutant following removal of ethylene is substantially slower than that observed for the wild type. The ethylene-insensitive mutants ETR1^{D25A}, ETR1^{D25E}, and ETR1^{D25Q} all exhibit ethylene insensitivity based on the short-term kinetic analysis (FIG. 6A).

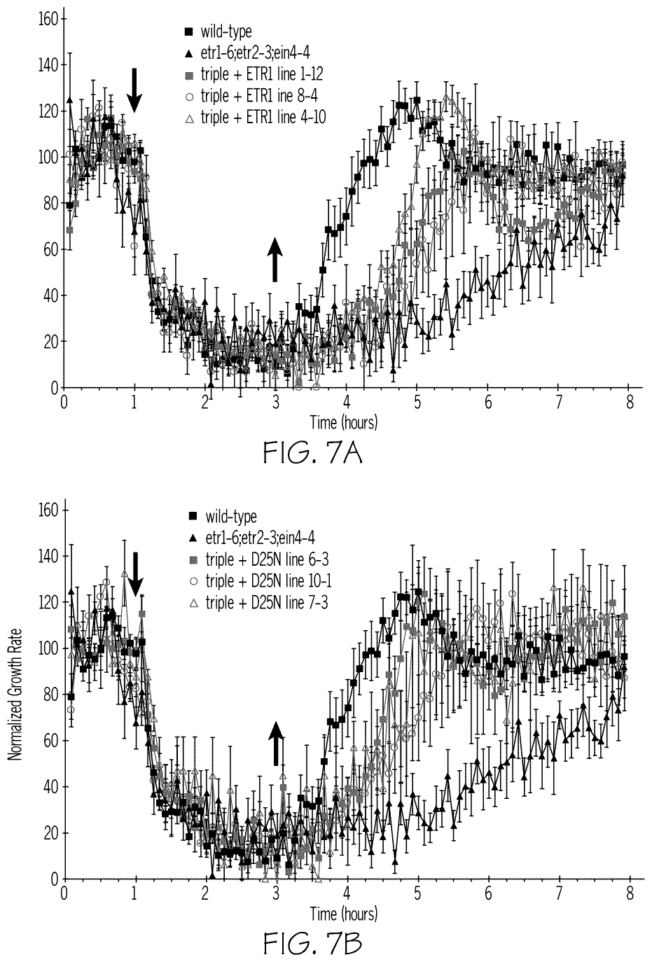

In contrast, both the ETR1^{wt} and ETR1^{D25N} lines exhibit a rapid growth response to ethylene and, and upon ethylene removal, a similar growth recovery intermediate between that exhibited by the wild-type and the etr1 etr2 ein4 seedlings (FIGS. 6A and 7A-7B). To examine the short-term molecular response, Applicant characterized ethylene-dependent gene expression for the ETR1 Asp25 mutants (FIG. 6B). Expression of OSR1 and ERF1 is induced, whereas the expression of EXP5 and EXPb1 is repressed, in response to ethylene. The molecular response to ethylene is similar in the ETR1^{wt} and ETR1^{D25N} transgenic lines for all four genes, and consistent with what is observed in the wild-type control. In contrast, the ethylene-insensitive mutants ETR1^{D25A}, ETR1^{D25E}, and ETR1^{D25Q} all exhibit various levels of hyposensitivity for ethylene-dependent gene expression, this being most apparent in the analysis of EXP5 and EXPb1 expression.

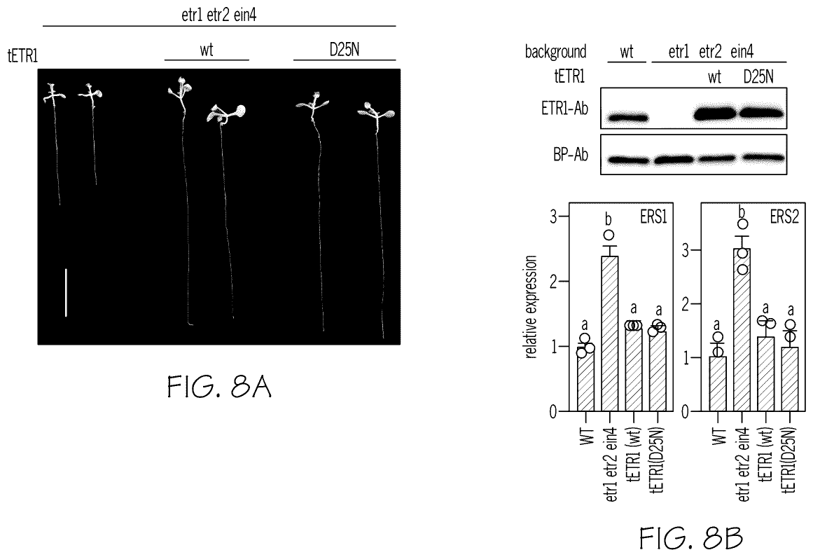

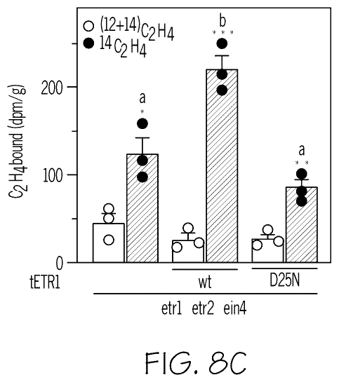

To determine whether ethylene binding of ETR1^{D25N} Arabidopsis lines mimicked what Applicant had observed using the heterologous yeast expression system, Applicant compared ethylene binding in planta of ETR1^{wt} and ETR^{D25N} in the etr1 etr2 ein4 background, making use of lines that exhibited a similar seedling phenotype, ETR1 protein levels, and expression levels of the remaining receptors ERS1 and ERS2 (FIGS. 8A-8B). [¹⁴C]ethylene binding was examined in two-week-old green seedlings grown on media containing 5 μM aminoethoxyvinylglycine (AVG) to inhibit ethylene biosynthesis. As shown in FIG. 8C, the triple mutant etr1 etr2 ein4 exhibits a basal level of ethylene binding due to the presence of the receptors ERS1 and ERS2, however transgenic expression of ETR1^{wt} results in a significant increase in ethylene binding. In contrast, no increase in ethylene binding was observed following transgenic expression of ETR^{D25N}.

### Example 1.3. Mechanism by which ETR1^{D25N} Mediates Ethylene Signaling in Planta

Without being bound by theory, Applicant considered two not mutually exclusive hypotheses as to how ETR1^{D25N} could mediate ethylene signaling in planta: residual ethylene binding of the ETR1^{D25N} mutant and/or cooperative interactions with other wild-type ethylene receptors in Arabidopsis. The first hypothesis is based on the affinity of the receptors for ethylene. ETR1 has a half-life for ethylene dissociation of over 12 hours, allowing for the ready detection of [¹⁴C]ethylene binding in yeast or in planta. However, if the ETR1^{D25N} mutant bound ethylene less tightly, then it would be substantially more difficult to detect with the [¹⁴C]ethylene binding assays. The second hypothesis is based on existence of ethylene receptor families in Arabidopsis. Even in the etr1;etr2;ein4 background, used for expression of the ETR1^{D25N} mutant, the ERS1 and ERS2 wild-type receptors are still present. These could bind ethylene and, as part of a receptor complex, potentially pass on a conformational information to ETR1(D25N), causing it to adopt a signaling conformation even when it has not bound ethylene. This type of signaling interaction has been found for bacterial chemoreceptors, and has also been proposed to occur for the ethylene receptors which, like chemoreceptors, are able to form higher-order receptor complexes.

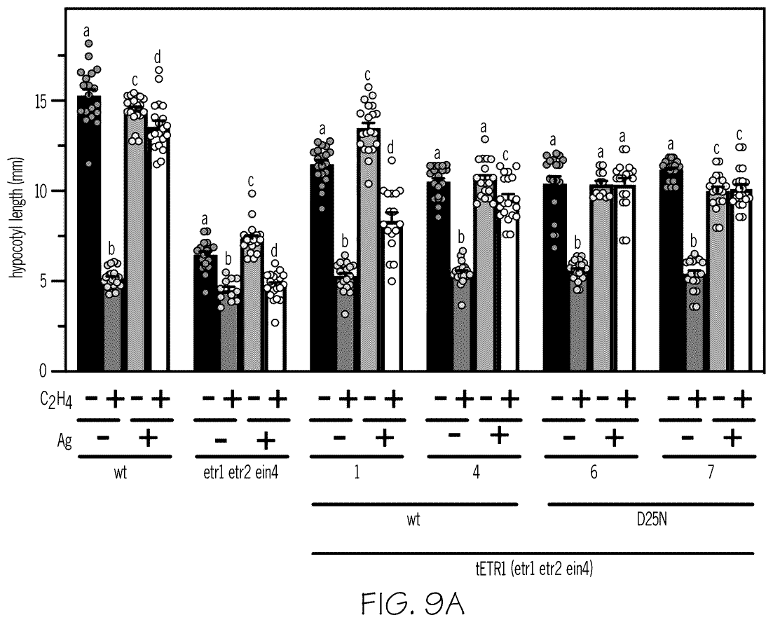

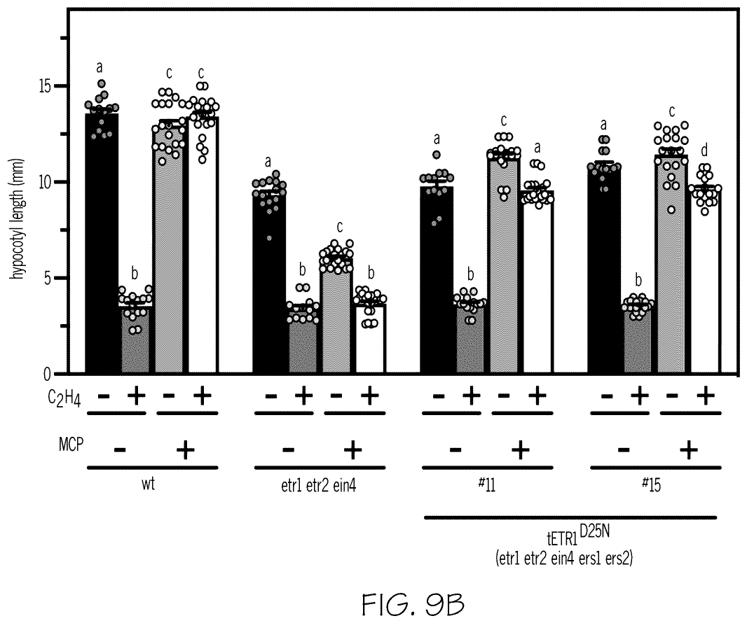

As an initial test for the feasibility of the first hypothesis, Applicant asked if ETR1^{D25}N could still bind a metal cofactor, and therefore potentially ethylene, in planta. For this purpose, Applicant examined the effects of silver (Ag) on ethylene sensitivity. Silver is thought to substitute for the copper cofactor and, although receptors containing silver can still bind ethylene, they no longer transmit the signal, resulting in ethylene insensitivity. As shown in FIG. 9A, the dark-grown seedling response of wild-type to 10 μL/L ethylene is blocked when seedling are grown on silver, but the etr1 etr2 ein4 triple mutant is still responsive to ethylene in the presence of silver. Applicant found that expression of either tETR1^{wt} or tETR1^{D25N} in the etr1 etr2 ein4 background restores the ability of silver to block the ethylene response, consistent with ETR1^{D25N} retaining an ability to bind its metal cofactor.

As an alternative approach to test the two hypotheses, Applicant used CRISPR-cas9 methodology to knock out ERS1 and ERS2 in the ETR1^{D25N} (etr1 etr2 ein4) line to generate an ETR1^{D25N} (etr1 etr2 ein4 ers1 ers2) line, the prediction being that signaling by ETR1^{D25N} will be lost if dependent on the presence of other wild-type receptors. Two independent ETR1^{D25N} (etr1 etr2 ein4 ers1 ers2) lines were generated (#11 and #15). However, contrary to the second hypothesis, the ETR1^{D25N} lines still responded to ethylene (FIG. 9B), consistent with an ability for ETR1^{D25N} to bind ethylene, in agreement with residual copper binding (FIG. 2B) and the silver sensitivity assay (FIG. 9A). Furthermore, the ethylene response of the ETR1^{D25N} (etr1 etr2 ein4 ers1 ers2) lines was blocked by the competitive inhibitor 1-methylcyclopropene (MCP) (FIG. 9B), also consistent with ETR1^{D25N} retaining its copper cofactor and ability to bind its gaseous ligand. These data thus support a key role for Asp25 of ETR1 in regulating the kinetics of ethylene binding by the receptor.

### Example 1.4. Effect of Lys91 Mutations of ETR1 on Seedling Growth and Ethylene Sensitivity

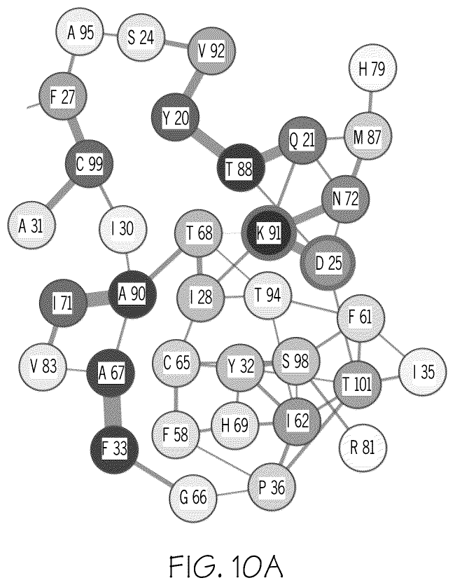

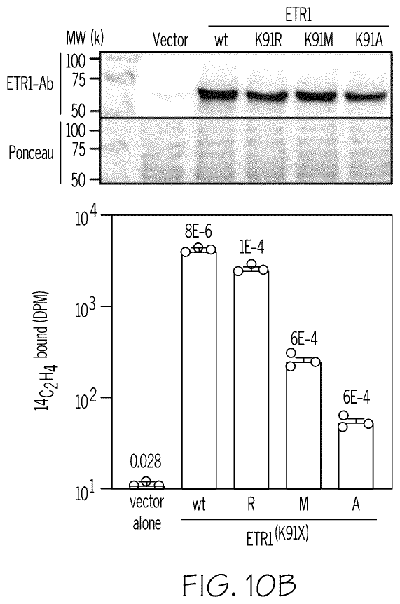

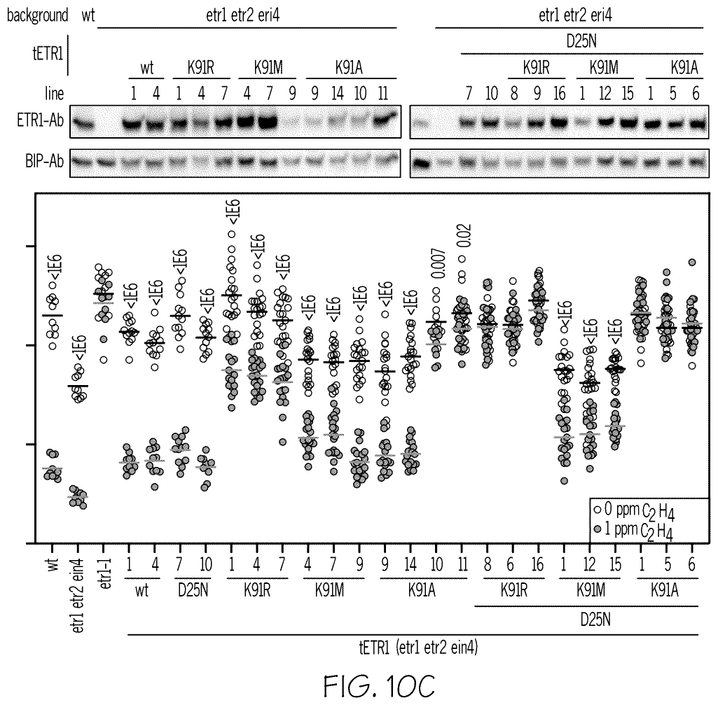

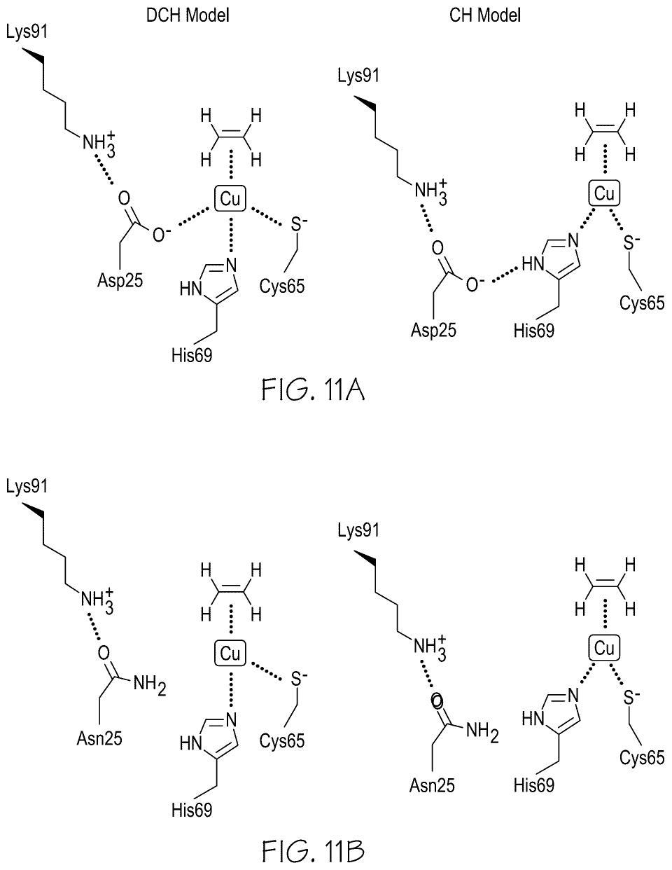

Sequence coevolution analysis predicts a coupling between Asp25 of TM helix I with Lys91 of TM helix III (GREMLIN probability of 0.960; EV couplings probability of 0.997; FIG. 10A). This predicted Asp25-Lys91 coupling is of interest because, in contrast to the first and second TM helixes of ETR1 that play substantial roles in copper and ethylene binding, the third TM helix is implicated in signal transduction. Therefore, a role for Asp25 in both copper binding and in forming a polar bond to TM helix III would provide a potential mechanism by which to couple ethylene binding to receptor signal output.

To examine the role of Lys91 in signaling by ETR1, Applicant made the site-directed mutations ETR1^{K91R}, ETR1^{K91M}, and ETR1^{K91A}, and examined their ethylene binding ability using the yeast expression system (FIG. 10B) as well as their functionality by transgenic expression in the etr1 etr2 ein4 Arabidopsis background (FIG. 10C). In ETR1^{K91R}, the basic Lys91 residue is replaced with another basic residue (Arg) which should preserve the ability to form a polar bond to Asp25. The ETR1^{K91R} mutant exhibited an ethylene binding ability similar to that of ETR1^{wt} (63% of wild-type binding) but like some other previously characterized site-directed mutations in transmembrane segment three, conferred ethylene hyposensitivity in Arabidopsis, potentially due to the larger Arg sidechain perturbing the receptor structure so that it does not effectively turn “off” upon ethylene binding.

In ETR1^{K91M}, the basic sidechain of Lys91 is replaced with a nonpolar sidechain of similar size; this mutation would no longer be able to participate in a polar bridge to mediate an ethylene-dependent change in conformation. ETR1^{K91M} exhibited substantially reduced binding of approximately 6% of that found in ETR1^{wt} (FIG. 10B). Interestingly, the ETR1^{K91M} mutant exhibited a seedling growth response not previously noted for ETR1 mutants. The ETR1^{K91M} mutant failed to rescue seedling growth efficiently but also exhibited partial ethylene hyposensitivity, suggesting that the receptor is less capable of maintaining the “on” and “off” conformations typically found in the absence and presence of ethylene, respectively, consistent with a lack of ‘communication’ between the ethylene binding site involving TM helixes I and II, and the output domain of TM helix III.

In ETR1^{K91A}, as with ETR1^{K91M}, the basic sidechain of Lys is replaced with a nonpolar sidechain but one that is smaller than that found with Lys; the ETR1^{K91A} mutation was previously found to confer ethylene insensitivity on Arabidopsis seedlings but still retained a low level of ethylene binding ability. Like ETR1^{K91M}, Applicant found that ETR1^{K91A} retained a minimal ability to bind ethylene (only 1% of that found in ETR1^{wt}). Thus, preservation of the basic nature of the Lys91 sidechain is important to ethylene binding, although the finding that substitution with a nonpolar sidechain does not eliminate ethylene binding is consistent with Lys91 not playing a direct role in chelating the copper cofactor. ETR1^{K91A} conferred two different ethylene response phenotypes in the seedling lines Applicant examined: a phenotype similar to that of the ETR1^{K91M} mutant or complete ethylene insensitivity such as previously reported for the mutation. The ETR1^{K91A} lines that exhibited ethylene insensitivity generally also had higher receptor protein levels, suggesting that insensitivity could arise due to increases in the number of misfolded receptors.

To gain further information on the interaction of Asp25 and Lys91, Applicant combined the Asp25Asn mutation with the ETR1^{K91X} mutations to generate ETR1^{D25N,K91R}, ETR1^{D25N,K91M} and ETR1^{D25N,K91A} and examined their functionality in the etr1 etr2 ein4 Arabidopsis background (FIG. 10C). The mutant combinations with Asp25Asn tended to accentuate the ethylene growth phenotypes noted for the ETR1^{K91X} single mutants. The ETR1^{D25N,K91R} mutant is more insensitive to ethylene than the ETR1^{K91R} mutant; the ETR1^{D25N,K91M} mutant exhibits a further reduction in the ability to rescue seedling growth and respond to ethylene compared to the ETR1^{K91M} mutant; and the ETR1^{D25N,K91A} mutant lines are all ethylene insensitive. The similarity of phenotypes for the ETR1^{D25N,K91X} mutants to those of the single ETR1^{K91X} mutants is consistent with Lys91 functioning ‘downstream’ of Asp25 in terms of signal transduction within the ETR1 transmembrane domain. The heightening of the ETR1^{K91X} phenotypes when combined with Asp25Asn also indicates that the Asp25Asn mutation does affect signaling by ETR1, even though the individual ETR1^{D25N} mutant could not be distinguished from the ETR1^{wt} by standard growth response assays (FIGS. 4A-4C and 6A-6B).

### Example 1.5. Discussion

A key but unresolved structural question for the ethylene receptors is how the copper cofactor(s) required for ethylene binding are coordinated within the TM domain. The Cu(I) oxidation state is known to exist in a variety of coordination geometries, with coordination numbers anywhere from two to six. Based on initial evidence for a single Cu(I) in the ethylene binding site, a tetrahedral geometry for the copper binding site was proposed involving Cys65 (as the thiolate form) and His69 of ETR1 (Cys65)₂(His69)₂, hereafter referred to as a CCHH coordination model. The tetrahedral geometry of the proposed CCHH copper-binding site was considered consistent with the homodimeric nature of ethylene receptors and the fact that Cys65 and His69 are on the same face of the second transmembrane helix, one helical turn apart.

Recent data point to the existence of two coppers per receptor dimer (i.e., one copper per receptor monomer), a possibility not inconsistent with the earlier study in which it was unclear if all the purified receptors were competent for copper binding, a finding that necessitates the consideration of new coordination structures for the copper cofactors. Here, Applicant implicate Asp25 of ETR1 as playing a critical role in copper binding based on the same principles that implicate Cys65 and His69. Computational modeling places Asp25 of helix I in proximity to Cys65 and His69 of helix II. Based on current understanding that there is one copper per monomer, we consider two potential models by which Asp25 could contribute to ethylene binding by ETR1 (FIGS. 11A-B). First, Asp25 could directly participate in copper binding along with Cys65 and His69 (a tridentate DCH model for copper coordination). Alternatively, Asp25 could form a hydrogen bond to His69 to orient and polarize it for copper binding (a bidentate CH model for copper coordination).

In addition to results from a recent in vitro study in which one copper per ETR1 monomer was detected, the DCH and CH models are favored over the earlier CCHH model for the following reasons. First, chemical analysis has demonstrated that both tridentate and bidentate ancillary ligands can effectively bind Cu(I) and form a Cu(I)-ethylene complex, the primary consideration being that anionic, electron-donating ancillary ligands foster the strongest backbonding of the filled Cu(I) 3d orbital to the unfilled ethylene π* orbital. Second, lower coordination numbers favor Cu(I) binding over that of Cu(II) and other divalent metal ions, supporting the existence of two or three copper-coordinating ligands, rather than four, with the ethylene receptors. Third, modeling of Cu(I) interactions with 1-methycyclopropene (1-MCP), a potent competitive inhibitor for ethylene binding, support Cu(I) being coordinated by no more than three ligands in addition to 1-MCP. Applicant note that, even should subsequent studies provide support for the CCHH model with one Cu(I) per receptor dimer, the role(s) for Asp25 identified here in copper binding are still relevant.

The CH model, in which the interaction of the Asp carboxylate with His contributes to Cu(I) binding may help explain the high binding affinity of the receptors for ethylene. Such carboxylate-His-metal interactions are fairly common in proteins, being a form of indirect carboxylate-metal coordination, with the carboxylate thought to modulate the histidine to make it a more effective Lewis base and strengthen the metal complexation. This carboxylate-His structure is similar to that found in the well-characterized ‘catalytic triads’ of serine proteases as well as in a host of other enzymes that also make use of an Asp-His interactions to facilitate hydrolytic and other enzymatic activities. The increase in the effectiveness of such enzymes may not just be due to increased nucleophilicity of the carboxylate-His, but also due to maintenance of the correct tautomer of His (e.g. N-1H vs N-3H) for the reaction, another consideration that may apply to the ability of His69 of ETR1 to interact with the Cu(I) cofactor.

An unexpected but physiologically relevant finding from Applicant's studies was that the substitution of Asn for Asp25 of ETR1, unlike the other site-directed mutations examined, still allowed for ethylene binding but affected the binding kinetics. Experimental analyses of [¹⁴C]ethylene binding in transgenic yeast and in planta are dependent on the extended half-life for dissociation of ethylene from the receptors, this being of over 12 hours for ETR1^{wt}. Applicant's inability to detect [¹⁴C]ethylene binding by ETR1^{D25N} when assayed in transgenic yeast suggests a half-life for dissociation on the order of minutes or less (k_{off} increasing at least 100-fold), the in planta recapitulation of the results from yeast indicating that this difference in ethylene binding between ETR1^{wt} and ETR1^{D25N} is not an artifact of the transgenic yeast system.

Interestingly, although Asp is found in 93.55% of the ETR1-like sequences examined (FIG. 2A), in some cases (3.67% of the sequences examined) it is substituted by an Asn residue, most commonly in cyanobacteria but also reportedly in the plants Pyrus communis (Pear) and Cajanus cajan (Pigeon pea). Applicant considers it likely that the Asn-containing ethylene receptor-like proteins of cyanobacteria facilitate chemotaxis., the ability to rapidly detect and respond to changes in ligand concentration not being compatible with the extended binding kinetics associated with the typical plant receptors. Plants, with their slower release kinetics for ethylene, rely upon proteasome-dependent degradation of ethylene-bound receptors and transcriptional induction of new receptors to facilitate resensitization once environmental ethylene levels decrease.

Based on Applicant's findings, Asp25 of ETR1 plays a dual role in signaling, functioning in copper/ethylene binding as well as in internally transmitting information on ethylene binding to TM helix III through an association with Lys91 (FIGS. 11A-11B). Coevolution analysis predicts a strong association between these two residues, both of which are highly conserved, the one residue acidic and the other basic, suggestive not only of physical proximity but the ability to make a strong polar bond. Most interesting was the reduced functionality in the absence and presence of ethylene observed with the ETR1^{K91M}, ETR1^{D25N,K91M} and some of the ETR1^{K91A} mutant lines, a phenotype consistent with a necessity to transmit conformational information from the ethylene binding site to TM helix III. These mutants may be at an equilibrium between the conformations typically found in the absence (“on”) and presence (“off”) of ethylene, with ethylene no longer able to stabilize one conformation over the other. Alternatively, if the receptors can take on intermediate conformations, the receptors may be stuck in such an intermediate conformation. Critically, the results of these mutations indicate the importance of the basic character of Lys91 in maintaining functionality of ETR1, and how loss of this character uncouples ETR1 from the conformations needed to mediate responses in the absence and presence of ethylene.

### Example 1.6. Plant Materials and Growth Conditions

All Arabidopsis lines were of the Columbia (Col-0) accession. The etr1-1 and etr1-6 etr2-3 ein4-4 mutant lines have been described. Analysis of the triple response of dark-grown Arabidopsis seedlings to ethylene was performed as described previously. Briefly, seedlings were grown at 22° C. on vertically oriented plates on half-strength Murashige and Skoog basal medium with Gamborg's vitamins (pH 5.75; Sigma), 0.8% (w/v) agar, and 5 μM aminoethoxyvinylglycine (AVG) to inhibit ethylene biosynthesis. The stratified seed was exposed to light for 8 hr and then moved to the dark in the presence or absence of ethylene, with analysis of the seedling growth being performed following 4 days total growth. For short-term kinetic analysis of dark-grown seedlings, time-lapse imaging and growth rate analysis of hypocotyls were carried out as described previously.

### Example 1.7. Generation of Site-Directed Mutations

ETR1 constructs used for expression in Arabidopsis were all derived from a 7.3-kb genomic ETR1 fragment containing the full-length coding sequence and native genomic promoter in the vector pCAMBIA1380. Site directed mutagenesis was performed according to the manufacturers with the QuikChangeII XL Site Directed Mutagenesis Kit (Agilent Technologies) for mutations of Asp25 or with the Q5 Site-Directed Mutagenesis kit (NEB) for mutations at other sites. For plant transformation, constructs were introduced into Agrobacterium tumefaciens strain GV3101 and transformed into the etr1-6 etr2-3 ein4-4 background by the floral-dip method. Lines containing single sites of insertion for the transgene were identified based on segregation of hygromycin resistance and brought to homozygosity for analysis.

ETR1 constructs used for expression in yeast were all derived from an ETR1 cDNA driven by the ADH1 promoter in the vector pYcDE-2. Site-directed mutations of Asp25 were introduced using the same primers and methodologies described above for expression of ETR1 in Arabidopsis. Mutations of Lys91 were introduced by replacing a Msc I-Sac I restriction fragment in the ETR1 cDNA with that from the genomic ETR1 mutant. Yeast constructs were transformed into the yeast Saccharomyces cerevisiae strain FY834 (MATα his3Δ200 ura3-52 leu2Δ1 lys2Δ202 trp1Δ63 GAL2+),

### Example 1.8. Generation of CRISPR-Cas9 Mutant Lines Targeting ERS7 and ERS2

To target ERS1 and ERS2, a tandem CRISPR cassette was synthesized that encoded four sgRNAs (two against ERS1 and two against ERS2) driven by U6 promoters and surrounded by HpaI and NaeI sites and cloned into pUC57 (General Biosystems). The guide RNAs were designed using CRISPR-P 2.0 to introduce indel mutations in the first exons of ERS1 and ERS2 that encode the ethylene binding site. The HpaI/NaeI fragment containing the gRNA cassette was cloned into the PmeI site of the pCAMBIA2300-Cas9 vector, which had been generated by taking the NsiI/KpnI restriction fragment with Cas9 from the plasmid pMTN3164 and cloning into the NsiI/PstI sites of pCambia2300 (GenBank™ accession no. AF234315). The CRISPR-ERS1/ERS2 plasmid was transformed into the Agrobacterium strain GV3101, and the Arabidopsis line ETR1^{D25N}-#6 (etr1 etr2 ein4) transformed by the floral dip method. Heat stress treatment of transgenic lines was used to increase the efficiency of CRISPR-Cas9 mutagenesis.

For identification and genotyping of CRISPR/Cas9 mutants, genomic DNA was isolated, and the region surrounding the CRISPR target sequence amplified by PCR and sequenced using various primers. The presence of the T-DNA insert containing the Cas9 cassette was determined by PCR using primers for the KanR gene. Two independent ETR1^{D25N} (etr1 etr2 ein4 ers1 ers2) lines were generated (#11 and #15), line #11 being Cas9 (−/−) and line #15 being Cas9(+/+).

### Example 1.9. Immunoblot Analysis

For yeast, total protein was extracted by bead-beating using a Mixer Mill 400 tissue homogenizer (Retsch). For plants, microsomes were isolated from seedlings. ETR1 was identified by use of a polyclonal anti-ETR1 antibody generated against amino acids 401-738 of ETR1. Immunoblot analysis was performed using an anti-BIP antibody or Ponceau-S staining for protein as loading controls. Immunodecorated proteins were visualized by chemiluminescence using the chemiDoc MP imaging system (BIORAD).

### Example 1.10. Copper Binding Assay

Copper binding was monitored spectrophotometrically by measuring absorbance of the purple BCA2-Cu(I) complex. Titration curves were corrected for nonspecific binding observed with chemically and thermally denatured receptor to maximize signal to background levels.

### Example 1.11. Ethylene Binding Assay

[¹⁴C]ethylene (specific activity=116 mCi/mmol) was obtained from ViTrax Radiochemicals (Placentia, CA) and trapped as the mercuric perchlorate complex as described. For ethylene binding assays in yeast, ETR1 was expressed in the yeast Saccharomyces cerevisiae (strain FY834) using the vector pYcDE-2 and a constitutive ADC1 promoter. The yeast growth media was supplemented with 40 μg L⁻¹ copper sulfate. Saturable ethylene binding to yeast was determined by analyzing binding of 0.3 g yeast per sample to 0.21 μL L⁻¹ [¹⁴C]ethylene, in the presence or absence of excess [¹²C]ethylene.

For ethylene binding assays with Arabidopsis seedlings, two-week-old green seedlings were used that had been grown on media containing 5 μl M AVG to inhibit ethylene biosynthesis, with ˜1 g seedlings per sample. The fresh weight of seedlings was determined, and each sample packaged into bags formed from a layer of cheese cloth and stapled at the top. Humidity was maintained in the ethylene binding chambers by use of moistened paper towels. Saturable ethylene binding was determined by analyzing binding to 0.31 μL L⁻¹ [¹⁴C]ethylene, in the presence or absence of excess [¹²C]ethylene.

### Example 1.12 RNA Expression Analysis

RNA isolation and RT-qPCR was performed with three biological replicates and two technical replicates of each.

### Example 1.13. Statistical Analysis

Unpaired T-tests were performed in Prism (GraphPad Software, Inc.), without the assumption of a consistent SD, to obtain the individual P values. ANOVA-based statistical analyses was performed using an online calculator.

### Example 1.14. Evolutionary Analysis to Predict Contacting Residues of ETR1

For the coevolutionary analysis, Applicant used EVCOUPLINGS and GREMLIN web servers to predict interactions/contacting residues in ETR1 ethylene binding domain. The 1-112 amino-acid sequence of ETR1 was used as an input for both servers. The EVCOUPLINGs algorithm derives residue-residue evolutionary couplings (ECs) from deep multiple sequence alignment by pseudo-likelihood maximization method. Applicant used the default settings for finding the evolutionary couplings between contacting residues where residue contact distance threshold was set to 5 and maximum rank was 1. For GREMLIN, the multiple sequence alignment was performed by HHBLITS and the alignment then filtered to remove regions where the gap was greater than 75.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. An ethylene sensor comprising:
an ethylene recognition component, wherein the ethylene recognition component comprises at least one ethylene binding domain, wherein the ethylene binding domain comprises:
an Asn residue,
at least one variant region that reduces the binding affinity of ethylene to the ethylene receptor when compared to the ethylene binding domain without the variant region,
at least one mutation that reduces the binding affinity of ethylene to the ethylene binding domain when compared to the ethylene binding domain without the mutation, or
combinations thereof; and
a detecting component, wherein the detecting component is operational to generate a detectable signal that correlates to the binding of ethylene to the ethylene binding domain.

2. The ethylene sensor of claim 1, wherein the ethylene recognition component is in the form of a layer.

3. The ethylene sensor of claim 1, wherein the ethylene recognition component comprises a lipid bilayer, wherein the ethylene binding domain is embedded in the lipid bilayer.

4. The ethylene sensor of claim 1, wherein the ethylene recognition component comprises a cultured cell, wherein the ethylene binding domain is associated with the cultured cell, and wherein the cultured cell contains or is associated with the detecting component.

5. The ethylene sensor of claim 4, wherein the ethylene binding domain is embedded with a lipid bilayer of the cultured cell.

6. The ethylene sensor of claim 4, wherein the cultured cell contains at least a portion of the detecting component.

7. The ethylene sensor of claim 6, wherein the detecting component comprises a tagged gene that is expressed in response to the binding of ethylene to the ethylene binding domain, and wherein the expressed protein of the tagged gene represents the detectable signal.

8. The ethylene sensor of claim 7, wherein the expressed protein comprises a fluorescent protein.

9. The ethylene sensor of claim 1, wherein the ethylene binding domain represents an isolated fragment of an ethylene receptor.

10. The ethylene sensor of claim 1, wherein the ethylene binding domain is part of an ethylene receptor.

11. The ethylene sensor of claim 9 or 10, wherein the ethylene receptor is selected from the group consisting of ETR1, ETR2, EIN4, ERS1, ERS2, ERS1, ERS2, ETR2, ETR3, ETR4, SynSLR1212 (SynETR1) of the cyanobacterium Synechocystis sp. PCC6803, analogs thereof, homologs thereof, or combinations thereof.

12. The ethylene sensor of claim 9 or 10, wherein the ethylene receptor originates from at least one of Arabidopsis thaliana, Oryza sativa, cyanobacteria, or combinations thereof.

13. The ethylene sensor of claim 9 or 10, wherein the ethylene receptor comprises ETR1.

14. The ethylene sensor of claim 9 or 10, wherein the ethylene receptor comprises SEQ ID NO: 1 or a sequence with at least 40% sequence identity to SEQ ID NO: 1.

15. The ethylene sensor of claim 9 or 10, wherein the ethylene receptor comprises SEQ ID NO: 1.

16. The ethylene sensor of claim 1, wherein the ethylene binding domain has a half-life for ethylene release of less than 12 hours.

17. The ethylene sensor of claim 1, wherein the ethylene binding domain has a half-life for ethylene release of less than 15 minutes.

18. The ethylene sensor of claim 1, wherein the ethylene binding domain comprises an Asn residue.

19. The ethylene sensor of claim 18, wherein the Asn residue represents a variant of the ethylene binding domain.

20. The ethylene sensor of claim 18, wherein the Asn residue represents a mutation of the ethylene binding domain.

21. The ethylene sensor of claim 18, wherein the mutation represents a mutation from Asp to Asn.

22. The ethylene sensor of claim 18, wherein the mutation comprises a mutation on a copper binding domain of the ethylene recognition component, wherein the mutation reduces the binding affinity of copper to the copper binding domain when compared to the binding affinity of copper to the copper binding domain without the mutation.

23. The ethylene sensor of claim 1, wherein the ethylene binding domain comprises at least one variant region that reduces the binding affinity of ethylene to the ethylene receptor when compared to the ethylene receptor without the variant region.

24. The ethylene sensor of claim 23, wherein the variant region comprises an Asn residue.

25. The ethylene sensor of claim 1, wherein the ethylene binding domain comprises at least one mutation that reduces the binding affinity of ethylene to the ethylene receptor when compared to the ethylene receptor without the mutation.

26. The ethylene sensor of claim 25, wherein the mutation comprises an Asn residue.

27. The ethylene sensor of claim 25, wherein the mutation represents a mutation from Asp to Asn.

28. The ethylene sensor of claim 1, wherein the ethylene binding domain comprises SEQ ID NO: 2 or a sequence that shares at least 40% sequence identity with SEQ ID NO: 2.

29. The ethylene sensor of claim 1, wherein the ethylene binding domain comprises SEQ ID NO: 3 or a sequence that shares at least 40% sequence identity with SEQ ID NO: 3.

30. The ethylene sensor of claim 1, wherein the ethylene binding domain comprises SEQ ID NO: 4 or a sequence that shares at least 40% sequence identity with SEQ ID NO: 4.

31. The ethylene sensor of claim 1, wherein the ethylene binding domain comprises SEQ ID NO: 5 or a sequence that shares at least 40% sequence identity with SEQ ID NO: 5.

32. The ethylene sensor of claim 1, wherein the detecting component is in the form of an electrode.

33. The ethylene sensor of claim 1, wherein the detecting component comprises a physicochemical transducer, wherein the physicochemical transducer is operational for converting at least one physicochemical change produced from the binding of ethylene to the ethylene receptor to the detectable signal.

34. The ethylene sensor of claim 33, wherein the physicochemical transducer is selected from the group consisting of an amperometric transducer, a potentiometric transducer, an impedance-based transducer, a conductometric-based transducer, an optical biotransducer, a field-effect transistor, a gravimetric transducer, a pyroelectric transducer, or combinations thereof.

35. The ethylene sensor of claim 1, wherein the detecting component is coupled to the ethylene recognition component.

36. The ethylene sensor of claim 35, wherein the detecting component is in electrical communication with the ethylene recognition component.

37. The ethylene sensor of claim 1, wherein the ethylene sensor further comprises a display, wherein the display is operational for displaying the detectable signal.

38. The ethylene sensor of claim 37, wherein the display comprises a digital screen.

39. The ethylene sensor of claim 37, wherein the display is in electronic communication with the detecting component.

40. The ethylene sensor of claim 1, wherein the detectable signal is selected from the group consisting of an optical signal, an electrical signal, a fluorescent signal, or combinations thereof.

41. The ethylene sensor of claim 1, wherein the detectable signal is proportionate to the concentration of the ethylene.

42. The ethylene sensor of claim 1, wherein the detectable signal is detectable in real-time.

43. The ethylene sensor of claim 1, wherein the ethylene sensor is operational to continuously sense ethylene in real-time.

44. A method of sensing an ethylene from an environment, said method comprising:
exposing the environment to the ethylene sensor of claim 1;
detecting the presence or absence of a signal from the ethylene sensor; and
correlating the absence of the signal to the absence of ethylene in the environment, or
correlating the presence of the signal to the presence of ethylene in the environment.

45. The method of claim 44, wherein the environment comprises an agricultural environment.

46. The method of claim 44, wherein the environment comprises a petrochemical environment.

47. The method of claim 44, wherein the ethylene is in the form of a gas.

48. The method of claim 44, wherein the sensing occurs continuously.

49. The method of claim 44, wherein the sensing occurs in real-time.

50. The method of claim 44, wherein the method is utilized to monitor the ripening of fruits or vegetables.

51. The method of claim 50, wherein the method is utilized to avoid the decomposition of the fruits or vegetables.

52. The method of claim 44, wherein the method is utilized to monitor the release of ethylene from a petrochemical plant.

53. A modified cell comprising:
at least one ethylene binding domain, wherein the ethylene binding domain comprises:
an Asn residue,
at least one variant region that reduces the binding affinity of ethylene to the ethylene binding domain when compared to the ethylene binding domain without the variant region,
at least one mutation that reduces the binding affinity of ethylene to the ethylene binding domain when compared to the ethylene binding domain without the mutation, or
combinations thereof, and
wherein the ethylene binding domain is expressed by at least one of an exogenous gene, a mutated gene, an over-expressed gene, or combinations thereof.

54. The modified cell of claim 53, wherein the ethylene binding domain is expressed as a component of an ethylene receptor.

55. The modified cell of claim 53, wherein the ethylene binding domain is expressed as a fragment that is separate and apart from an ethylene receptor.

56. The modified cell of claim 53, wherein the ethylene binding domain is expressed as part of a chimeric receptor.

57. The modified cell of claim 53, wherein the modified cell comprises a bacterial cell.

58. The modified cell of claim 53, wherein the modified cell comprises a plant cell.

59. The modified cell of claim 53, wherein the modified cell comprises a seed cell.

60. The modified cell of claim 54 or 55, wherein the ethylene receptor is selected from the group consisting of ETR1, ETR2, EIN4, ERS1, ERS2, ERS1, ERS2, ETR2, ETR3, ETR4, SynSLR1212 (SynETR1) of the cyanobacterium Synechocystis sp. PCC6803, analogs thereof, homologs thereof, or combinations thereof.

61. The modified cell of claim 54 or 55, wherein the ethylene receptor originates from at least one of Arabidopsis thaliana, Oryza sativa, cyanobacteria, or combinations thereof.

62. The modified cell of claim 54 or 55, wherein the ethylene receptor comprises ETR1.

63. The modified cell of claim 54 or 55, wherein the ethylene receptor comprises SEQ ID NO: 1 or a sequence with at least 40% sequence identity to SEQ ID NO: 1.

64. The modified cell of claim 54 or 55, wherein the ethylene receptor comprises SEQ ID NO: 1.

65. The modified cell of claim 53, wherein the ethylene binding domain comprises an Asn residue.

66. The modified cell of claim 65, wherein the Asn residue represents a variant of the ethylene binding domain.

67. The modified cell of claim 65, wherein the Asn residue represents a mutation of the ethylene binding domain.

68. The modified cell of claim 67, wherein the mutation represents a mutation from Asp to Asn.

69. The modified cell of claim 53, wherein the ethylene binding domain comprises at least one variant region that reduces the binding affinity of ethylene to the ethylene binding domain when compared to the ethylene binding domain without the variant region.

70. The modified cell of claim 69, wherein the variant region comprises an Asn residue.

71. The modified cell of claim 53, wherein the ethylene binding domain comprises at least one mutation that reduces the binding affinity of ethylene to the ethylene binding domain when compared to the ethylene binding domain without the mutation.

72. The modified cell of claim 71, wherein the mutation comprises an Asn residue.

73. The modified cell of claim 71, wherein the mutation represents a mutation from Asp to Asn.

74. The modified cell of claim 71, wherein the mutation comprises a mutation on a copper binding domain of the ethylene recognition component, wherein the mutation reduces the binding affinity of copper to the copper binding domain when compared to the binding affinity of copper to the copper binding domain without the mutation.

76. A modified plant or seed, wherein the modified plant or seed comprises the modified cell of claim 53.


SEQUENCE LISTING (7 sequences)

SEQ ID NO: 1 - protein, 738 aa, Arabidopsis thaliana
MEVCNCIEPQWPADELLMKYQYISNFFIAIAYFSIPLELIYFVKKSAVFPYRWVLVQFGAFIVLCGATHLINLWTFTTHSRTVALVMTTAKVLTAVVSCATALMLVHIIPDLLSVKTRELFLKNKAAELDREMGLIRTQEETGRHVRMLTHEIRSTLDRHTILKTTLVELGRTLALEECALWMPTRTGLELQLSYTLRHQHPVEYTVPIQLPVINQVFGTSRAVKISPNSPVARLRPVSGKYMLGEVVAVRVPLLHLSNFQINDWPELSTKRYALMVLMLPSDSARQWHVHELELVEVVADQVAVALSHAAILEESMRARDLLMEQNVALDLARREAETAIRARNDFLAVMNHEMRTPMHAIIALSSLLQETELTPEQRLMVETILKSSNLLATLMNDVLDLSRLEDGSLQLELGTFNLHTLFREVLNLIKPIAVVKKLPITLNLAPDLPEFVVGDEKRLMQIILNIVGNAVKFSKQGSISVTALVTKSDTRAADFFVVPTGSHFYLRVKVKDSGAGINPQDIPKIFTKFAQTQSLATRSSGGSGLGLAISKRFVNLMEGNIWIESDGLGKGCTAIFDVKLGISERSNESKQSGIPKVPAIPRHSNFTGLKVLVMDENGVSRMVTKGLLVHLGCEVTTVSSNEECLRVVSHEHKVVFMDVCMPGVENYQIALRIHEKFTKQRHQRPLLVALSGNTDKSTKEKCMSFGLDGVLLKPVSLDNIRDVLSDLLEPRVLYEGM

SEQ ID NO: 2 - protein, 100 aa, Arabidopsis thaliana
WPADELLMKYQYISDFFIAIAYFSIPLELIYFVKKSAVFPYRWVLVQFGAFIVLCGATHLINLWTFTTHSRTVALVMTTAKVLTAVVSCATALMLVHIIP

SEQ ID NO: 3 - protein, 100 aa, Arabidopsis thaliana
WPADELLMKYQYISNFFIAIAYFSIPLELIYFVKKSAVFPYRWVLVQFGAFIVLCGATHLINLWTFTTHSRTVALVMTTAKVLTAVVSCATALMLVHIIP

SEQ ID NO: 4 - protein, 95 aa, Synechocystis sp. PCC 6803
WQTPLVWLHVSADFFTAIAYYSIPLTLLYFLRKRQDIPFPNIIFLFSTFILCCGTSHFFDIITLWYPIYWISGTVKASMAIVSIITVFELIQIVP

SEQ ID NO: 5 - protein, 95 aa, Stenomitos frigidus
WQTPLVGLHVVSNALIAIAYFSIPAMLVYFVHKRQDVPFSKVFIMFGAFIVLCGTGHLLDIWTLWHPAYWLAGGVRALTALVSCYTALQLLTLLP

SEQ ID NO: 6 - protein, 98 aa, unidentified
YLWQPGLVWLHVISDALIALAYFSIPLALIYFVRKRRDLPFRWVFLLFGAFIVLCGTTHLLEIWTLWHPSYWLSGLVKALTALVSVATAIALVPLIPK

SEQ ID NO: 7 - protein, 98 aa, unidentified
WPADELLMKYQYISDFFIAIAYFSIPLELIYFVKKSAVFPYRWVLVQFGAFIVLCGATHLINLWTFTTHSRTVMTTAKVLTAVVSCATALMLVHIIPD